(19) United States
(12) Patent Application Publication
(10) Pub. No.: US 2026/0259200 A1
(43) Pub. Date: Sep. 3, 2026

(54) VANISHING DEVICE FOR ENRICHMENT, RETRIEVAL, SEPARATION, ARRANGEMENT OF TARGETED AGENTS AND METHODS OF FABRICATION

(71) Applicant: GEORGIA TECH RESEARCH CORPORATION, Atlanta, GA (US)

(72) Inventors: Ali Fatih SARIOGLU, Atlanta, GA (US); Mert BOYA, Atlanta, GA (US); Tevhide Ozkaya AHMADOV, Atlanta, GA (US)

(21) Appl. No.: 18/994,814

(22) PCT Filed: Jul. 17, 2023
(86) PCT No.: PCT/US2023/027905

§ 371 Date: Jan. 15, 2025

Related U.S. Application Data
(60) Provisional application No. 63/389,717, filed on Jul. 15, 2022.

(51) Int. Cl. G01N 33/537; G01N 1/40

(52) U.S. Cl. CPC G01N 33/5375; G01N 1/4077

(57) ABSTRACT

Vanishing device or structure made of specifically-linked hydrogel that can be configured, via the linkage chemistry, for a number of different applications, including enrichment, retrieval, separation, and/or arrangement of a targeted agent within an environment and then to, additionally, fully dissolve under a pre-defined condition such as heat, body temperature, light, time, among other examples described herein for that application. The separation, and subsequent vanishing, allows for the isolation and/or enrichment of the targeted agent for subsequent analysis that would otherwise damage, degrade, or affect the target agent of interest. The separation can also arrange or retrieve the target agent in a structure that can then vanish to allow for the subsequent analysis of the target agent.

### CROSS-REFERENCE TO RELATED APPLICATIONS

This application claims the benefit of priority to U.S. Provisional Application No. 63/389,717, filed Jul. 15, 2022, entitled “Vanishing devices for enrichment, retrieval, and analysis of target particles,” which is incorporated by reference herein in its entirety.

### BACKGROUND

Fluid biopsy or fluid phase biopsy is the sampling and analysis of non-solid biological tissue or agent, primarily in blood. A sample is typically acquired and directly analyzed for the presence of a target agent of interest, which can include cancer cells, diseased cells, DNA, or other biological cells released by cancer cells or other abnormal biological processes. Fluid biopsy, or similar processes, can be performed on cerebrospinal fluid, urine, and other body fluids for the target agent.

When DNA analysis is desired, polymerase chain reaction (PCR) is performed on a sample, which is often first subjected to centrifuge or lysis processing for isolation of the DNA material.

Microfluidic devices are one class of devices that are employed to arrange or classify biological samples, e.g., to isolate cells or particles of interest. Microfluidic devices can include chemistry functionalization (e.g., biological or chemical coating) or electrical functionalization (via electrical fields, e.g., dielectrophoresis), to assist with the isolation and/or classification of the cell or particles of interest.

There is a benefit to improving the separation and enrichment of biological target agents and inorganic compounds of interest.

### SUMMARY

An exemplary vanishing device or structure (also interchangeably referred to herein as a sacrificial device or structure) is disclosed made of specifically-linked hydrogel that can be configured, via the linkage chemistry, for a number of different applications, including enrichment, retrieval, separation, and/or arrangement of a targeted agent within an environment and then to, additionally, fully dissolve under a pre-defined condition such as heat, body temperature, light, time, among other examples described herein for that application. The separation, and subsequent vanishing, allow for the isolation and/or enrichment of the targeted agent for subsequent analysis that would otherwise damage, degrade, or affect the target agent of interest. The separation can also arrange or retrieve the target agent in a structure that can then vanish to allow for the subsequent analysis of the target agent.

Isolation refers to the setting apart of a targeted agent from other agents in its native (i.e., sampled) environment. Enrichment refers to the isolation of the targeted agent to a higher concentration meaningful to a downstream analysis. Retrieval refers to the setting apart of a targeted agent from other agents in its native environment for the purpose of extracting the targeted agent from that native/sampled environment. Arrangement refers to the arranging or classifying of a targeted agent in the structure (e.g., microfluidic or fluidic device) to which the structure can then vanish to allow for the subsequent analysis (or manufacturing or other operation described herein).

An exemplary method of fabrication for the various examples is also described in addition to a method to fabricate the various devices that employs multiple stages using semiconductor processing (to form silicon master dies) and hydrogel processing operations (that employs the master dies to form one or more stages of sacrificial polymer-based dies) to fabricate such devices in high volume and at low costs.

The exemplary vanishing device, in one example, as a liquid biopsy device, is configured via a functionalization, geometry, or chemistries, e.g., for isolating, by capturing and retaining, tumor or diseased cells shed into a body fluid, as a sample in the sample holder, that then can be subsequently extracted in a fully sacrificial manner that does not damage the retained tumor cells. The device can begin dissolving based on time, normal body/physiological temperature, or external stimuli such as light, among other stimuli provided in the examples herein. A similar sacrificial device can be employed to isolate other types of cells or particles of interest, as described in the various examples herein.

The exemplary vanishing device, as a polymerase chain reaction (PCR) tool for extracting cells for a PCR analysis, in another example, is configured for isolating and separating cells or biological material or interest in front-end operation (e.g., before centrifuge or lysis) of an integrated PCR test device that then can be subsequently extracted in a fully sacrificial manner that does not damage the retained cells or biological material. The device can begin dissolving based on time or external stimuli such as light or heat (typical PCR processes are often performed at elevated temperatures to those of normal body/physiological temperature).

The exemplary vanishing device, as a microfluidic device or a component thereof, can be formed as or with a scaffold structure that can retain particles in neighboring features such as wells and then later dissolved (i.e., vanished or sacrificed), e.g., to bring the particles together to form bonds, e.g., tissue engineering or tissue manufacturing. The exemplary vanishing device, according to this implementation, can be employed for manufacturing and can be used for separating, and arranging targets having pH in the range of 4-10. This can be employed for inorganic applications.

The exemplary vanishing device, as another example, can be formed having a hybrid structure that includes a sacrificial/vanishing component and a non-sacrificial/non-vanishing component. For example, in one implementation, the vanishing device is a cover structure for a non-vanishing container. This can also be employed for inorganic applications.

In one aspect, a sacrificial device is provided that can be used to enrich, retrieve, separate, arrange, or a combination thereof, a target agent and then subsequently fully or substantially dissolve as a vanishing or sacrificial structure to provide the target agent for subsequent analysis or processing (e.g., manufacturing). In some aspects, the sacrificial device may comprise a hydrogel that forms a sacrificial structure. In some aspects, the sacrificial structure can be configured to retain and/or separate a targeted agent (such as a particle or a cell) within an environment. In some aspects, the sacrificial device may be configured to release the targeted agent when subjected to a releasing condition. In some aspects, the releasing condition can be compatible with the targeted agent and deconstructs the sacrificial structure.

In another aspect, a method of isolating a targeted agent from a sample is provided. In some aspects, the method can comprise contacting the sample with a sacrificial device (such as a sacrificial device described herein). In some aspects, the sacrificial device can bind the targeted agent. In some aspects, the method can further comprise separating via the sacrificial device the targeted agent from the sample. In some aspects, the method can further comprise subjecting the sacrificial device to a releasing condition. In some aspects, the targeted agent may be released and the sacrificial device completely or substantially sacrificed (such as at least 50%).

In a further aspect, a method of fabricating a vanishing device is provided. In some aspects, the method may comprise fabricating a first mold. In some aspects, the first mold can be made in part of silicon and patterned using a silicon micromachining and/or manufacturing process. In some aspects, the method can further comprise generating a second mold using the first mold (such as a PDMS mold). In some aspects, the method can further comprise generating the vanishing device from the second mold or a third mold derived therefrom.

The details of one or more aspects of the disclosure are set forth in the accompanying drawings and the description below. Other features, objects, and advantages of the disclosure will be apparent from the description and drawings and from the claims.

### DESCRIPTION OF DRAWINGS

FIG. 1 shows examples of sacrificial devices made of hydrogel forms a fully or semi-fully sacrificial structure configured to either retain, retrieve, arrange, separate, or a combination thereof, a targeted agent within a liquid environment and then to be sacrificed under a pre-defined condition to release the targeted agent in accordance with an illustrative embodiment.

FIGS. 2A-2G each show example configurations of vanishing devices that can retain, retrieve, and/or separate a target agent in accordance with an illustrative embodiment.

FIG. 3 shows a vanishing device that can be employed for inorganic applications in accordance with an illustrative embodiment.

FIG. 4A shows an example method of isolating a targeted agent from a sample in accordance with an illustrative embodiment.

FIG. 4B shows a method for fabricating a vanishing device in accordance with an illustrative embodiment.

FIGS. 5A-5B depict a working example of a sacrificial biochip as an exemplary sacrificial device described herein. FIG. 5A shows a schematic illustration of the device operation. The sacrificial biochip arrests the target cells while letting non-target cells pass into the waste. The device then dissolves on demand at an elevated temperature and leaves the isolated cells behind. FIG. 5B (top-left) shows a photo of a microfabricated sacrificial biochip being held by a tweezer. Scale bar, 5 mm. FIG. 5B (bottom-left) shows a close-up fluorescence microscope image of the device showing an isolated tumor cell cluster lodged in one of the trapezoidal pores. FIG. 5B (top-right) shows a fluorescence microscope image showing the cross-section of a trapezoidal pore with a single tumor cell captured in it. FIG. 5B (bottom-right) shows a plot showing the flow streamlines, simulated using finite element analysis, in the vicinity of a trapezoidal pore in the sacrificial biochip. Scale bars, 10 μm. FIG. 5C (top) shows a schematic illustration of the fabrication process for micropatterning gelatin within a PDMS mold subsequent to the silicon micromachining and the manufacturing of the PDMS mold (see Example 1: Methods; FIG. 10). FIG. 5C (bottom) shows scanning electron micrographs of the micromachined silicon mold (left) and the final device microfabricated out of gelatin (right). Scale bars, 10 μm. FIG. 5D shows time-lapse photos showing the dissolution of the sacrificial biochip, which was fluorescently labeled for visualization purposes. The inserts show close-up microscope images of the sacrificial biochip showing the transformation of trapezoidal pore geometry during the heat-induced dissolution process. Scale bars, 20 μm.

FIGS. 6A-6C further depicts a representative working principle of the sacrificial device. FIG. 6A shows a schematic illustration of the sacrificial device's working principle. While passing healthy cells and other constituents of body fluids to the waste due to their larger size, tumor cells are retained on the device for further downstream analyses, including fluorescence imaging, in vitro cell culture, and molecular analysis. FIG. 6B shows time sequence photographs of the vanishing process of a device in 1× PBS by increasing the temperature of the solution to 37° C. The device was colored with a green dye for visual illustration. FIG. 6C shows microscopy images of the device that show the structural details during the vanishing process. A device with 25 mm diameter takes ~4 min to fully vanish at 37° C. Scale bars, 20 μm.

FIGS. 7A-7B depict representative design features which may be applied to the sacrificial devices. FIG. 7A shows different representative pore shapes and sizes which may be used. FIG. 7B shows different representative structural designs which may be used.

FIGS. 8A-8B depict various design choices that can be used for clustered particles. FIG. 8A shows different pore shapes and sizes. FIG. 8B shows different structural designs.

FIGS. 9A-9C depict an exemplary embodiment of a microfluidic sacrificial device. FIG. 9A shows patterned gelatin chips. FIG. 9B shows a microscope image of patterned microfluidic channels. FIG. 9C shows a photo of a microfluidic sacrificial device.

FIG. 10 depicts the fabrication process of the sacrificial biochip. A schematic illustration of the fabrication process is shown, which involves silicon micromachining, PDMS replication, and vacuum-assisted micro-molding.

FIG. 11 depicts the water contact angle measurement of the gelatin-based device. The measurement was performed by dropping a small water droplet on the fabricated gelatin device. Image processing was performed using ImageJ software for measuring the water contact angle. The contact angle is measured as ~60.7°.

FIG. 12 depicts the effect of polar organic solvents on the device. Microscope images of the device incubated in DMSO and glycerol for 30 minutes at room temperature. The structural change in the device showed the incompatibility of the device in polar organic solvents. Scale bars, 20 μm.

FIGS. 13A-13F depict the characterization of the sacrificial biochip. FIG. 13A shows representative photos and microscope images of dry and wet devices. The pore size of dry devices was designed by considering the swelling property of gelatin. Scale bars, 5 mm. FIG. 13B shows the swelling ratio of the device in solutions with different pH levels (n=3 independent experiments). Data are presented as mean ±SD. FIG. 13C shows further swelling of devices during their operation in various liquid biopsy sources. Minimal swelling was observed in addition to the initial swelling in 1× DPBS solution (n=3 independent experiments). Data are presented as mean ±SD. FIG. 13D shows the autofluorescence of devices compared to commercially available polycarbonate and polyester filter membranes under four different excitation/emission wavelengths (n=3 independent experiments). Data are presented as mean ±SD. FIG. 13E shows non-specific protein binding levels of the gelatin, polycarbonate, and polyester filter membranes when incubated with Cy-5 conjugated IgG protein (n=3 independent experiments). Data are presented as mean ±SD. FIG. 13F shows the time required for fully dissolving the sacrificial biochip with respect to the solution temperature (n=3 independent experiments). Data are presented as mean ±SD.

FIG. 14 depicts the dissolution time of unstrengthened devices with respect to the solution temperature. Following the fabrication, devices were placed inside pre-heated 1× PBS solutions at different temperatures without any prior incubation with 1× DPBS buffer containing divalent Mg²⁺ and Ca²⁺ ions for strengthening the material. Significantly lower dissolution times were observed compared to the devices incubated in DPBS solution for all temperatures tested (n=3 independent experiments). Data are presented as mean ±SD.

FIGS. 15A-15F depict tumor cell enrichment using the sacrificial biochip. FIG. 15A shows a schematic showing the process of preparing the device for installation into a filter holder. The device, swollen in 1× DPBS, is attached to an O-ring that mechanically constrains the device against collapse and facilitates ease of handling. FIG. 15B shows a scanning electron micrograph of a CSF-spiked ONS-76 tumor cell that is captured by the sacrificial biochip. Scale bar, 10 μm. FIG. 15C shows a plot showing tumor cell capture efficiencies of devices when operated at different flow rates. The experiments were performed by spiking LNCaP human prostate tumor cells into unprocessed whole blood samples. (n=3 independent experiments). Data are presented as mean ±SD. FIG. 15D shows a plot reporting white blood cell retention rates of the sacrificial biochip and commercial polycarbonate filter when both processed 10 mL of whole blood at the same flow rate of 10 mL/h. The gelatin device achieved ~10× lower WBC counts remaining on the device following the PBS wash (n=3 independent experiments). Data are presented as mean ±SD. FIG. 15E shows a representative fluorescence image of a captured LNCaP tumor cell cluster on the device. Following the blood filtration and PBS wash, isolated cells were stained on the device using FITC-conjugated antibodies against EpCAM and prostate-specific membrane antigen (PSMA) and imaged using a fluorescence microscope. Scale bar, 10 μm. FIG. 15F shows representative images of fixed, stained images of an ONS-76 human medulloblastoma tumor cell isolated from aCSF sample and T24 human bladder tumor cell cluster isolated from a urine sample using the sacrificial biochip. The ONS-76 tumor cells were stained with Synaptophysin, NCAM, B7-H3 (green), and DAPI (nuclei, blue), and the T24 tumor cells were stained with Cytokeratin 8/18 (green) and DAPI (nuclei, blue). Scale bars, 10 μm.

FIG. 16 depicts a comparison of near-surface hydrodynamic flow fields between the device and a conventional membrane filter. Plots show flow field streamlines along with flow speed simulated using finite element analysis. The slanted walls of the sacrificial biochip (top) direct the flow into pores minimizing the stagnant flow regions compared to a conventional membrane filter with a flat surface topography (bottom).

FIGS. 17A-17E depict downstream analysis of spiked tumor cells. FIG. 17A shows a representative illustration of the release process that utilizes the heat-activated dissolving property of sacrificial biochip. Once the temperature is increased to physiological rates, the device dissolves in minutes, leaving the isolated cells for further processing. FIG. 17B shows the percentage viability of control, processed, and released cells into a Petri dish (n=3 independent experiments). The two-color viability test was performed on the control and released cells inside the Petri dishes (see Methods), which were subsequently scanned using a fluorescence microscope and counted to calculate the percentage of viable cells. Data are presented as mean ±SD. FIG. 17C shows the short-term culture of ONS-76 cells following the isolation and release processes. The cell media was changed every 3 days for a period of 10 days. Scale bars, 10 μm. FIG. 17D shows a schematic illustration of the process flow applied for molecular analysis of isolated cells on the sacrificial biochip. Following the 1× PBS wash, the device is directly folded and placed inside a PCR tube with a lysis buffer. FIG. 17E shows AdnaTest RT-PCR results on the samples analyzed with Agilent 2100 Bioanalyzer. Shown gel electropherograms illustrate (i) the reliable operation of the assay with positive and negative controls (ii) the effect of presence of device with cells for gel electrophoresis, (iii) the dependence of the assay signal on the number of cells, (iv) the effect of contaminating cells. The sizes of the detected bands are: PSMA: 449 bp, PSA: 357 bp, EGFR: 163 bp, and Actin: 111 bp, where PSMA, PSA and EGFR were used as tumor-specific targets and Actin was used as an internal control.

FIG. 18 depicts testing the viability of cells enriched using the sacrificial biochip. Fluorescence microscope images of the control (left) and chip-processed (right) medulloblastoma human tumor cells (ONS-76) after they were subjected to a two-color live (green)/dead (red) assay. The medulloblastoma cells spiked into aCSF sample was processed at 10 mL/h, and the isolated cells were released by dissolving the device at 37° C. Scale bars, 20 μm.

FIGS. 19A-19B depict the isolation and molecular analysis of patient tumor cells. FIG. 19A shows fluorescence microscope images of the captured cells from blood samples of (i) a prostate cancer patient, (ii) an ovarian cancer patient, and a CSF sample of (iii) a medulloblastoma patient. Captured prostate tumor cells were stained against Cytokeratin 8/18, PSA/KLK3, EpCAM (green), CD45 (red), and DAPI (nuclei, blue), ovarian tumor cells were stained against Cytokeratin 7, Cytokeratin 8/18, Vimentin (green), CD45 (red) and DAPI (nuclei, blue), and medulloblastoma cells were stained against Synaptophysin, NCAM, B7-H3 (green), CD45 (red) and DAPI (nuclei, blue). Scale bars, 10 μm. FIG. 19B shows gel electropherograms corresponding to results from multiplex RT-PCR analysis of CTCs isolated from prostate cancer patients. Primers used were specifically targeting tumor-specific markers i) PSMA (449 bp), PSA (357 bp), EGFR (163 bp), and ii) AR (440 bp). Actin (111 bp) was used as the internal control of the test kit. For both figures, the ladder is shown in the first lane while the negative and positive controls are shown in the last two lanes.

Like reference symbols in the various drawings indicate like elements. Variations of feature may share similar reference symbols.

### DETAILED DESCRIPTION

The following description of the disclosure is provided as an enabling teaching of the disclosure in its best, currently known aspects. Many modifications and other aspects disclosed herein will come to mind to one skilled in the art to which the disclosed devices and methods pertain, benefiting from the teachings presented in the descriptions herein and the associated drawings. Therefore, it is understood that the disclosures are not limited to the specific aspects disclosed and that modifications and other aspects are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As apparent to those of skill in the art upon reading this disclosure, each of the individual aspects described and illustrated herein has discrete components and features that may be readily separated from or combined with the features of any of the other several aspects without departing from the scope or spirit of the present disclosure.

Any recited method can be carried out in the order of events recited or any other order that is logically possible. Unless otherwise expressly stated, it is in no way intended that any method or aspect set forth herein be construed as requiring that its steps be performed in a specific order. Accordingly, where a method claim does not explicitly state in the claims or descriptions that the steps are to be limited to a particular order, it is in no way intended that an order be inferred in any respect. This holds for any possible non-express basis for interpretation, including logic concerning an arrangement of steps or operational flow, meaning derived from grammatical organization or punctuation, or the number or type of aspects described in the specification.

All publications mentioned herein are incorporated by reference to disclose and describe the methods or materials in connection with which the publications are cited. The publications discussed herein are provided solely for their disclosure before the filing date of the present application. Nothing herein is to be construed as an admission that the present invention is not entitled to antedate such publication by the prior invention. Further, the dates of publication provided herein can be different from the actual publication dates, which can require independent confirmation.

It is also to be understood that the terminology herein describes particular aspects only and is not intended to be limiting. Unless defined otherwise, all technical and scientific terms herein have the same meaning as commonly understood by one of ordinary skill in the art to which the disclosed compositions and methods belong. It can be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Before describing the various aspects of the present disclosure, the following definitions are provided and should be used unless otherwise indicated. Additional terms may be defined elsewhere in the present disclosure.

As used herein, “comprising” is interpreted as specifying the presence of the stated features, integers, steps, or components but does not preclude the presence or addition of one or more features, integers, steps, components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, “consisting essentially of” is intended to include examples encompassed by the term “consisting of.”

As used in the specification and the appended claims, the singular forms “a,” “an,” and “the” include plural referents unless the context dictates otherwise.

Ratios, concentrations, amounts, and other numerical data can be expressed herein in a range format. Further, the endpoints of each of the ranges are significant both in relation to the other endpoint and independently of the other endpoint. There are many values disclosed herein, and each value is also disclosed as “about” that particular value in addition to the value itself. For example, if the value “10” is disclosed, then “about 10” is also disclosed. Ranges can be expressed herein as from “about” one particular value and to “about” another particular value. Similarly, when values are expressed as approximations, using the antecedent “about,” the particular value forms a further aspect. For example, if the value “about 10” is disclosed, then “10” is also disclosed.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x,’ ‘about y,’ and ‘about z’ as well as the ranges of ‘less than x,’ ‘less than y.’ and ‘less than z.’ Likewise, the phrase ‘about x, y, z, or greater’ should be interpreted to include the specific ranges of ‘about x,’ ‘about y,’ and ‘about z’ as well as the ranges of ‘greater than x,’ greater than y,2 and ‘greater than z.’ In addition, the phrase “about ‘x’ to ‘y’,” where ‘x’ and ‘y’ are numerical values, includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5% but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, amounts, sizes, formulations, parameters, and other quantities and characteristics are not and need not be exact but may be approximate, larger or smaller, as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In such cases, as used herein, “about” and “at or about” mean the nominal value indicated ±10% variation unless otherwise indicated or inferred. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, “optional” or “optionally” means that the subsequently described event or circumstance can or cannot occur. The description includes instances where said event or circumstance occurs and those where it does not.

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed of “adjacent” another feature can have portions that overlap or underlie the adjacent feature.

Spatially relative terms, such as “under,” “below,” “lower,” “over,” “upper,” and the like, may be used herein for ease of description to describe an element's or feature's relationship to another element(s) or feature(s) as illustrated herein. It will be understood that the spatially relative terms are intended to encompass different orientations of the device in use or operation in addition to the orientation depicted herein. For example, if the device is inverted, elements described as “under” or “beneath” other elements or features would then be oriented “over” the other elements or features. Thus, the exemplary term “under” can encompass both an orientation of over and under. The device may otherwise be oriented (e.g., rotated 90 degrees or at other orientations), and the spatially relative descriptors used herein are interpreted accordingly. Similarly, the terms “upwardly,” “downwardly,” “vertical,” “horizontal,” and the like are used herein for the purpose of explanation only unless specifically indicated otherwise.

It should be understood that although the terms first, second, etc., may be used herein to describe various elements, components, regions, layers, and/or sections, these elements, components, regions, layers, and/or sections should not be limited by these terms. Rather, these terms are only used to distinguish one element, component, region, layer, and/or section from another element, component, region, layer, and/or section. Thus, a first element, component, region, layer, or section discussed herein could be termed a second element, component, region, layer, or section without departing from the teachings of the present disclosure. The sequence of operations (or steps) is not limited to the order presented in the claims or examples unless specifically indicated otherwise.

Compounds are described using standard nomenclature. Unless defined otherwise, all technical and scientific terms used herein have the same meaning as is commonly understood by one of skill in the art to which this disclosure belongs.

### Example Vanishing Devices with Sacrificial Structures

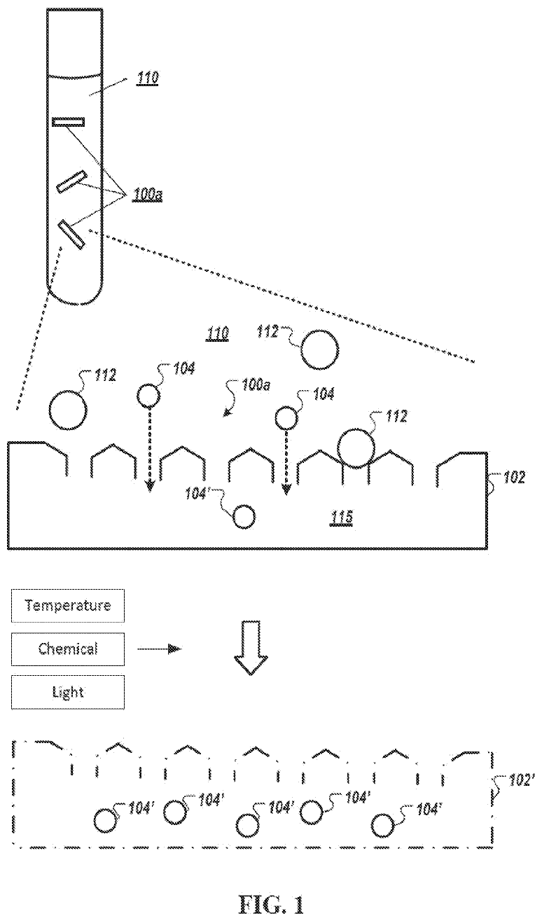

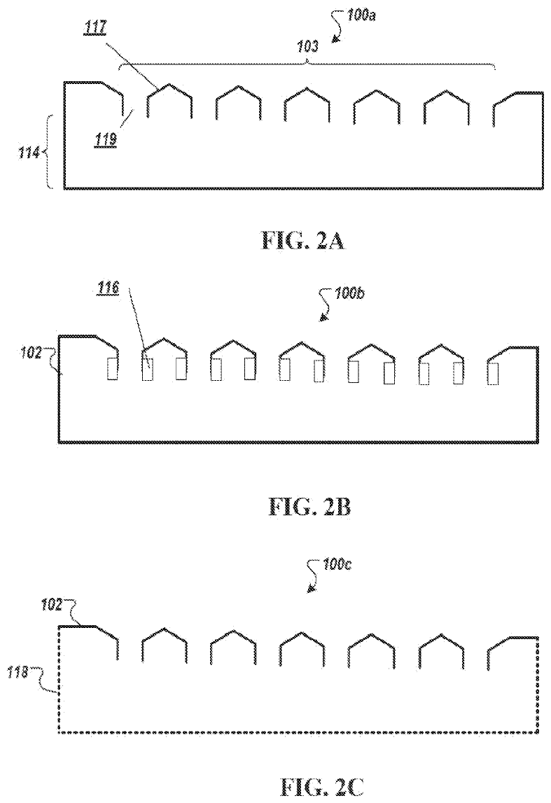

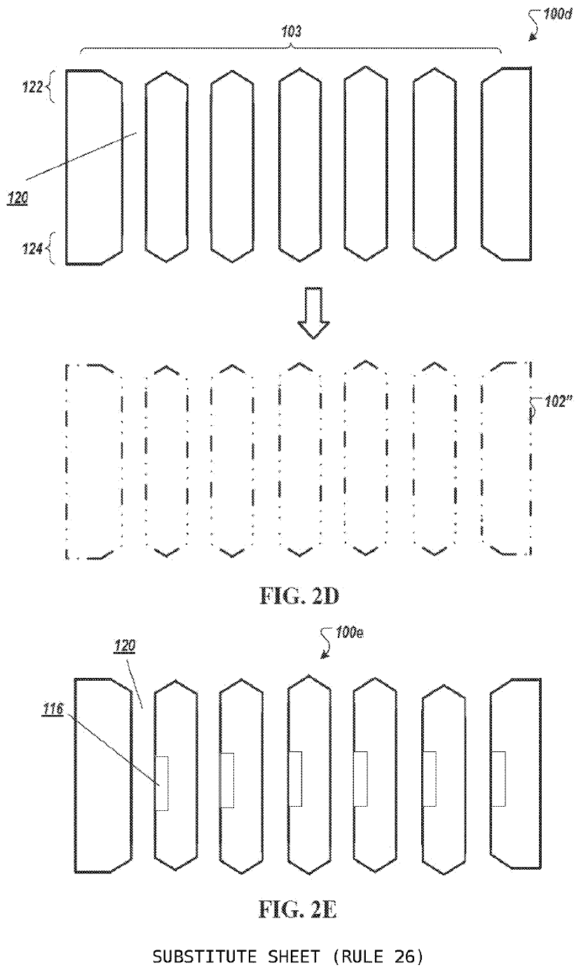

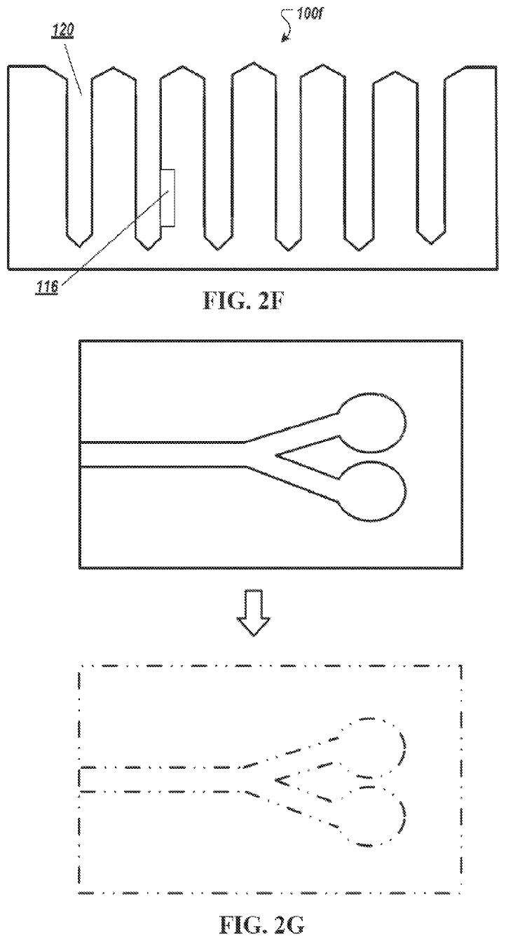

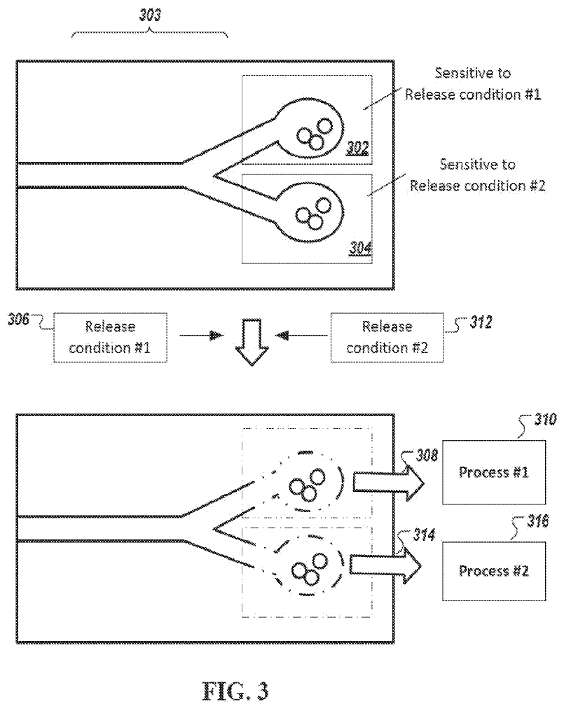

FIG. 1 shows examples of vanishing devices 100 made of hydrogel forms a fully or semi-fully sacrificial structure 102 having a topology (e.g., pattern) 103 that is configured to either retain, retrieve, separate, or a combination thereof, a targeted agent within a liquid environment and then to fully dissolve or semi-fully (e.g., 50%) under a pre-defined condition to release the targeted agent. FIGS. 2A-2G each show example configurations of vanishing devices that can retain, retrieve, and/or separate a target agent. FIG. 3 shows an example configuration of a vanishing device to retain, retrieve, arrange, and/or separate.

In the example shown in FIG. 1, the vanishing device 100 (shown as 100a) includes a structure 102 configured with a pattern 103 (shown as 103a) that can (i) retain and/or separate a targeted agent 104 (e.g., a particle or a cell) within an environment 106, by separating the target agent 104 from other non-target agent, and then (ii) release the targeted agent 104 when subjected to a releasing condition 108 compatible with the targeted agent 104 and by deconstruction of the sacrificial structure 102, shown as deconstructed structure 102′. The vanishing device 100a, in the example of FIG. 1, is shown having been placed in a sample environment 110, optionally, along with other sacrificial devices 100a, to isolate and/or enrich the concentration of the targeted agent 104 (shown as 104′ in its isolated or enriched form) from other agents 112 in the sample environment 110. The sample environment may be bodily fluid acquired from a subject and may include blood plasma, cerebrospinal fluid, or urine. In some implementations, the sample environment may be a manufacturing process having a pH level between 4 and 10.

In being able to fully deconstruct the sacrificial structure, the vanishing device 100 can be implemented, as shown in the examples herein, with complex topologies and/or chemical or electrical functionalizations that losslessly isolate, enrich, and/or arrange the target agent of interest and without impacting its structure or chemistry that could otherwise impact the downstream analysis or processing of the target agent.

Hydrogels are biphasic materials comprising a mixture of porous, permeable solids and typically at least 10% by weight or volume of an interstitial fluid composed completely or mainly of water. The porous, permeable solids are typically insoluble three-dimensional networks of natural or synthetic polymers and a fluid, having absorbed a large amount of water. In some aspects, the hydrogel can be biologically compatible if necessary for the particular application, such as retaining targeted agents from biological materials. Representative examples of hydrogels that can be used include, but are not limited to, hyaluronic acid, chitosan, heparin, alginate, gelatin, fibrin, polyvinyl alcohol, polyethylene glycol, sodium polyacrylate, acrylate polymers, and copolymers thereof. In particular aspects, the hydrogel includes gelatin.

In some implementations, the sacrificial hydrogel is crosslinked, e.g., ionically crosslinked, via Mg²⁺ and/or Ca²⁺ ions, to be sensitive to normal physiological temperatures. The cross-linked hydrogel can be ionically crosslinked via a suitable ion, such as cations or anions, or crosslinker which may be cleaved under a suitable condition such as when in contact with heat (i.e., a medium having elevated temperature to the physiological temperature), light, or a chemical reagent.

The vanishing device 100 is configured to release the targeted agent when subjected to a releasing condition that is compatible with the targeted agent; that is, the releasing condition (be it time, physiological temperature and environment, elevated temperature, or chemical, as described herein) is intended to not affect the targeted agent when the deconstruction (full or partial) of the vanishing sacrificial structure 102.

The targeted agent (e.g., 102) includes a biological agent, e.g., cluster of particles or cells, or inorganic material such as a particle or compounds isolated by the sacrificial structure 102. Representative examples of biological agents which may be isolated include, but are not limited to, cells, proteins, nucleic acids, and the like. Suitable cells include, but are not limited to, plant cells (e.g., monocot, dicot), animal cells (e.g., mammalian, avian, amphibian, reptile cells), or microbial cells (e.g., prokaryote, eukaryote, protozoal, etc.). The cells may be of differentiated cells from or corresponding to any type of tissue (e.g., blood, cartilage, bone, muscle, endocrine gland, exocrine gland, epithelial, endothelial, etc.), or may be undifferentiated cells such as stem cells or progenitor cells. In some embodiments, the cells may comprise cancer cells. Proteins as targeting agents may include any protein or peptide which may be bound by the binding agent, including fragments thereof, analogs thereof, and/or homologs thereof. Protein targets include proteins or peptides having any biological functional or activity, including structural, regulatory, hormonal, enzymatic, genetic, immunological, contractile, storage, transportation, and signal transduction. The protein target may include, in some embodiments, structural proteins, receptors, enzymes, cell surface proteins, proteins pertinent to the integrated function of a cell, including proteins involved in catalytic activity, aromatase activity, motor activity, helicase activity, metabolic processes (anabolism and catabolismantioxidant activity, proteolysis, biosynthesis, proteins with kinase activity, oxidoreductase activity, transferase activity, hydrolase activity, lyase activity, isomerase activity, ligase activity, enzyme regulatory activity, signal transducer activity, structural molecule activity, binding activity (for protein, lipid, or carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport including protein transporter activity, nuclear transport, iron transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity, or translation regulator activity. Protein targets of interest can include proteins from eukaryotes and prokaryotes, including microbes, viruses, fungi and parasites, including humans, microbes, viruses, fungi and parasites, among numerous others, including other animals, including domesticated animals, microbes, plants, and viruses.

In some implementations, the sacrificial device may be configured to isolate the targeted agent from an organism (e.g., by being placed within the organism) or a targeted agent within the natural environment, e.g., wetland, petroleum well, underground rivers.

The vanishing devices, in some embodiments, are compatible with a downstream analysis of the targeted agent, such as immunofluorescent imaging, RNA sequencing, polymerase chain reactions (including multiplex PCR), and functional assays such as cell culture, drug testing, or viability assays.

FIG. 2A shows the example vanishing device 100a of FIG. 1 formed wholly of a sacrificial structure 102 having a pattern 103 formed of the sacrificial structure 102 that separates a target agent 104 from other non-target agents. The vanishing device 100a includes a retaining portion 114 also formed of the sacrificial structure 102. The sacrificial structure 102 with the pattern 103 can be configured as contoured surface 117 and inlet regions 119, e.g., that can urge the targeted agent to a retaining portion or structure of the vanishing device 100.

In the example shown in FIG. 2A, the sacrificial structure 102 is configured to form a chamber 115 as a retaining portion 114. The example of FIG. 2A shows the chamber 115 has a number of inlet regions 117 formed in the contour surface. The multiple inlet regions 119 may be of the same size or may have multiple sizes or functionalizations (for example, two or three) in which a given size and/or functionalization can allow entry of certain targeted agents. Different structures and/or functionalization may be employed within a given pattern 103 that can isolate and enrich different target agents.

FIG. 2B shows the example vanishing device 100 (shown as 100b) of FIG. 2A also formed almost wholly of a sacrificial structure 102 with additional embedded functionalization region 116, e.g., a binding agents dispersed within or bound to (e.g., covalently or non-covalently linked to) the hydrogel. The binding agent binds the targeted agent to the hydrogel. In some aspects, the binding agent may be covalently or non-covalently linked to the hydrogel. In some aspects, the binding agent may be linked to the hydrogel via covalent bonding, physisorption, ionic bonding, hydrogen bonding, hydrophobic interactions, electrostatic interactions, van Der Waals interactions, or combinations thereof. Representative examples of binding agents include, but are not limited to, antibodies, antigens or fragments thereof, DNA or RNA aptamers, lectins, and small molecule or protein/peptide ligands for protein targets (such as receptors).

FIG. 2C shows an example vanishing device 100 (shown as 100c), as a hybrid structure, formed having a wholly sacrificial structure 102 (shown as 102a) that is fixably attached (e.g., via heating process, adhesive, or solvent bonding) to a non-sacrificial structure 118. The hybrid vanishing device 100c may incorporate the embedded functionalization region 116 as described in relation to FIG. 2B.

FIG. 2D shows an example vanishing device 100 (shown as 100d) formed wholly of a sacrificial structure 102 having a pattern 103 formed of the sacrificial structure 102 at two or more portions of the device 100d that can separate a target agent 104 from other non-target agents. The vanishing device 100a forms a set of channels 120 that can retain the target agent 104. The pattern 103 is formed at a first region 122 and a second region 124. Similar to the example shown in FIG. 1, the example vanishing device 100d is configured to fully deconstruct or functionally deconstruct, as shown by deconstructed structure 102″, to release any target agent retained in the channels 120.

FIG. 2E shows the example vanishing device 100d (shown as 100e) of FIG. 2D also formed almost wholly of a sacrificial structure 102 with additional embedded functionalization region 116, e.g., a binding agent such as an antibody, antigens, or other agents described in other examples herein.

FIG. 2F shows an example vanishing device 100 (shown as 100f) formed wholly of a sacrificial structure 102 having a pattern formed of the sacrificial structure 102 at a portion of the device 100d that can separate a target agent 104 from other non-target agents. The vanishing device 100f also forms a set of channels 120 that can retain the target agent 104. Similar to the example shown in FIG. 2F, the vanishing device 100f may include an embedded functionalization region 116 (shown for one of the channels).

FIG. 2G shows an example vanishing device 100 (shown as 100g) formed as a microfluidic device. The device can be configured for dielectrophoresis or include structures to direct the flow of target agents 104 for their separation and/or classification.

FIG. 3 shows a vanishing device that can be employed for inorganic applications. The vanishing device 300 includes a scaffold that can retain a target agent in neighboring structures such as wells and then dissolve the structure to bring particles together to form bonds. Such vanishing devices may be employed for tissue engineering or manufacturing.

In the example shown in FIG. 3, the target agents are separated, via region 303 into one of multiple vanishing structures 100 (shown as 302, 304), e.g., of a microfluidic device. The vanishing structure 302 is sensitive to a first release condition, and the vanishing structure 304 is sensitive to a second release condition. After sorting, the first release condition #1 (306) is applied to dissolve the vanishing structure 302. In doing so, the target agent of the first vanishing structure 302 is released (308) from the vanishing structure and allowed to be processed in a first process (310).

Subsequently, the second release condition #2 (312) is then applied to dissolve the vanishing structure 304. The target agent of the second vanishing structure 304 is released (314) from the vanishing structure and allowed to be processed in a second process (316).

### Example Releasing Conditions of the Sacrificial Device

As discussed above, the vanishing device (e.g., 100a-100g) is configured to be fully or substantially dissolvable under a pre-defined condition such as heat, body temperature, light, and time, among other examples described herein for that application. FIG. 1 shows the example releasing condition being shown applied to an exemplary vanishing device 100 to cause it to deconstruct and/or dissolve.

Temperature-induced device vanishing/deconstruction. The releasing condition may comprise the sacrificial structure 102 or device 100 being heated when the hydrogel is temperature or heat sensitive. In some implementations, the hydrogel can melt upon heating of the sacrificial structure 102 or device 100. Heating as a releasing condition may occur at a pre-defined temperature to release the targeted agent, e.g., from about 35 degrees Celsius to about 38 degrees Celsius, corresponding to a physiological temperature. In other particular implementations, heating may occur at an elevated temperature to those of physiological temperature, for example, at a denaturation, annealing, or extension temperature typically used in a polymerase chain reaction, e.g., from about 45 degrees Celsius to about 100 degrees Celsius.

Light-induced device vanishing/deconstruction. In other aspects, the releasing condition may comprise the sacrificial device being contacted with light when the hydrogel is light-sensitive. Light-sensitive hydrogels may be formed by the utilization of light-sensitive crosslinkers or via light-sensitive moieties present with a polymer backbone of the hydrogel.

Chemical-induced device vanishing/deconstruction. In further aspects, the releasing condition may comprise exposing the sacrificial device to a chemical treatment. The chemical treatment may comprise contacting the sacrificial device with a reagent capable of cleaving one or more moieties present in the hydrogels and/or associated crosslinkers. In some particular aspects, the chemical treatment may comprise an enzymatic treatment. In such instances, the crosslinkers for the hydrogel may comprise peptide crosslinkers.

### Example Method of Operation

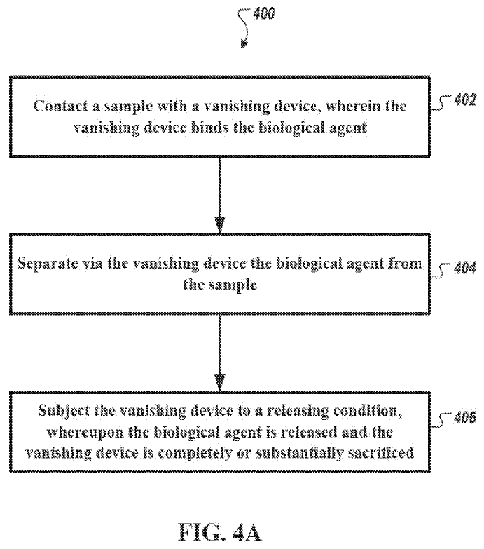

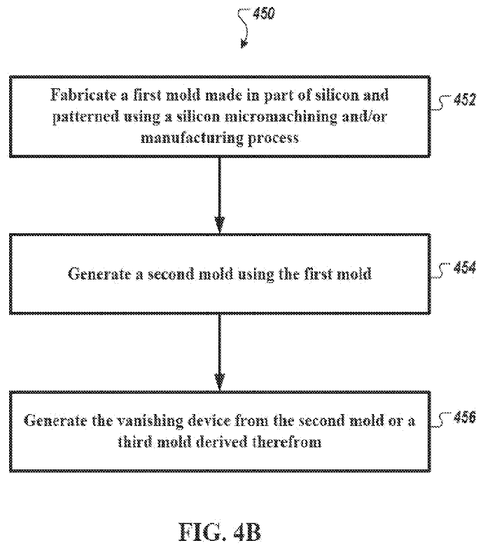

FIG. 4A shows an example method 400 of isolating a targeted agent from a sample. In the example shown in FIG. 4A, the method 400 includes contacting (402) the sample with a sacrificial device 100 (e.g., 100a-100g) in which the sacrificial device 100 binds the targeted agent. The sacrificial device 100 is configured as a filter or a separator that can separate the target agent (e.g., 104) from the sample or other agents through the sacrificial structure (e.g., 102).

Method 400 then includes subjecting (404) the sacrificial device to a releasing condition, whereupon the targeted agent (e.g., 104) is released from the sacrificial device (e.g., 100) when it is completely or substantially sacrificed.

The sacrificial device (e.g., 100) may be subjected to any suitable releasing condition appropriate to the sacrificial device as has been described herein. In some aspects, subjecting the sacrificial device to a releasing condition may comprise heating the vanishing device or an environment of the vanishing device, wherein the releasing condition of the vanishing device is heat sensitive. In other aspects, subjecting the sacrificial device to a releasing condition may comprise illuminating the sacrificial device or an environment of the sacrificial device, wherein the releasing condition of the sacrificial device is light-sensitive. In other aspects, subjecting the sacrificial device to a releasing condition may comprise contacting the sacrificial device or an environment of the sacrificial device with a reagent, wherein the releasing condition comprises a chemical treatment with the reagent.

The sacrificial device may bind the targeted agent via a binding agent as described herein dispersed within or bound to the sacrificial device.

### Example Method of Fabrication

FIG. 4B shows a method 450 for fabricating a vanishing device described herein. In the example, the method 450 includes fabricating (452) a first mold. The first mold may be made in part of silicon and patterned using a silicon micromachining and/or manufacturing process.

Method 450 then includes generating (454) a second mold using the first mold. In some implementations, the second mold may comprise a PDMS mold.

Method 450 may then include generating (456) the vanishing device from the second mold or a third mold derived therefrom.

The first mold may include a contoured surface to form the contour surface of the fabricated vanishing device that urges a biological agent to a retaining portion of the vanishing device when in an environment, e.g., as described in relation to FIGS. 2A-2F or other examples described herein. The contoured surface of the first mold may be dimensioned according to a pre-defined swelling ratio or dimensional adjustments for when the fabricated vanishing device is in the environment.

In an example, the method 450 may use a 3-step molding process in which two steps are employed to create plastic (PDMS) molds and one step to fill the channels of the second PDMS structure. Indeed, it is optional to create two or more stages of plastic (PDMS) molds. The second mold is structurally the same as the first mold just different material. The use of a second mold can help preserve the first mold out of silicon. In other words, each molding step can create a complement geometry of the mold. The second mold can give the geometry of the original mold that can be directly used, e.g., filled in its channels with hydrogel, to fabricate the end product.

In other embodiments, the first mold can be used to create the first mold as the complement of the original geometry. In that case, only one step is required to create the plastic mold for filling.

Additional examples are described later herein.

### EXPERIMENTAL EXAMPLES

The following examples are put forth to provide those of ordinary skill in the art with a complete disclosure and description of how compositions, articles, devices, and methods claimed herein are made and evaluated and are intended to be purely exemplary of the invention and are not intended to limit the scope of what the inventors regard as their invention. Efforts have been made to ensure accuracy concerning numbers (e.g., amounts, temperature, etc.), but some errors and deviations should be accounted for. Unless indicated otherwise, parts are parts by weight, the temperature is in degrees Celsius or is at ambient temperature, and pressure is at or near atmospheric pressure.

Assaying tumor cells shed into bodily fluids offers the potential for non-invasive detection and analysis of cancerous tissue. Harvesting tumor cells shedding into bodily fluids, known as liquid biopsy, facilitates the analysis of cancerous tissues from specimens that could be acquired more frequently and less invasively than conventional surgical biopsies [1,2]. Tumor cells present in peripheral blood [3], ascites [4-6], cerebrospinal fluid (CSF) [7-9], and urine [10-13] of cancer patients have all been sought after for studies on metastasis of various solid tumors with the ultimate goal of correlating clinical outcomes with information that can be retrieved from those tumor cells. Therefore, technologies that can reliably deliver these breakaway tumor cells in a state amenable for interrogation are vitally needed for their eventual translation into clinical use for personalized cancer treatments.

Despite remarkable technological advances in sensitive detection and enumeration of rare tumor cells admixed with other cells, recovery of those tumor cells at their native state, amenable for further analysis, remains a challenge. The focus has long been a technique's sensitivity and/or specificity for detecting tumor cells against a background of non-tumor cells in collected specimens [14,15]. The fact that tumor cells are only found at very low concentrations and are highly heterogenous justifies employing sample manipulation schemes that utilize or amplify any contrast, physical and/or chemical, between tumor cells and the rest. Recent development of microchip-based assays further enabled precise manipulation of individual cells and employed innovative discrimination schemes to detect a single tumor cell among billions of normal healthy cells [16-18].

On the other hand, those liquid biopsy tools and associated sample manipulation schemes, all assets during the enrichment of tumor cells from a specimen, often end up being liabilities downstream when those enriched cells are to be analyzed. The effects of the cell enrichment process on analyses are most detrimental if the isolated tumor cells are to be assayed beyond enumeration. For example, capturing tumor cells with immobilized antibodies offers specificity at the expense of challenges in the recovery of these cells from the device post-processing [19-25]. Likewise, fixation of tumor cells for size-based enrichment prevents larger tumor cells from squeezing through smaller pores even if they were compliant to do so in their native state, but limits use of cells for functional studies [26]. Magnetic beads attached to cells were sought to be detached from labeled cells once they were enriched for a variety of assays [27-30]. Even continuous-flow, label-free enrichment methods employ buffer solutions for enrichment and lead to low-concentration cell suspensions that potentially require lossy concentration processes for analyses [31-33].

Described herein is a vanishing cell isolation device that, when desired, dissolves to give unimpeded access to isolated cells for analyses. The ability for the whole cell enrichment apparatus to disappear from an assay offers significant advantages over existing cell isolation techniques that strive to discharge enriched cells from a structure designed to retain those same cells in the first place. Rather than exposing the captured cells to mechanical stresses [25] or harsh chemicals such as proteolytic digestion enzymes [48], which ultimately affect their viability, sacrificial biochips dissolve at physiological temperatures, passively releasing viable cells that can proliferate. In addition, unlike commonly employed sacrificial coating strategies [17,49,50] that expose released cells to potential recapture or entrapment on their way out that decrease yield, a fully dissolved sacrificial biochip ensures guaranteed retrieval of each and every cell on the device. Finally, passively releasing stationary cells by dissolving the retaining structure eliminates the need for shearing buffer flow to detach and carry cells, effectively providing a non-diluted population that can be subjected to any assay without lossy concentration and resuspension steps. Convenience and ease of use of the vanishing device, which allow bypassing the need for staining, scanning, and micromanipulation of target tumor cells, open new avenues for the utilization of isolated tumor cells in research and clinical settings.

### Exemplary Sacrificial Device

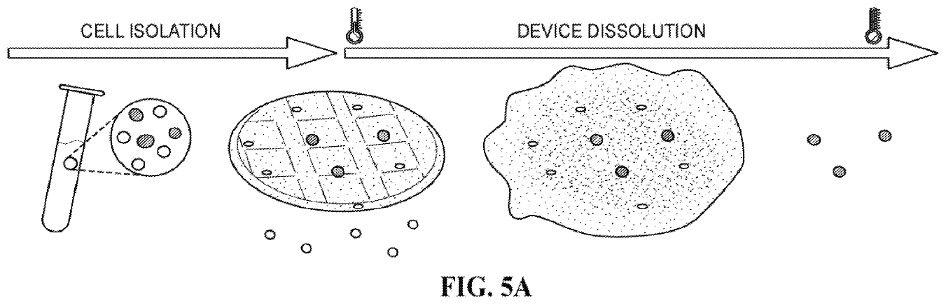

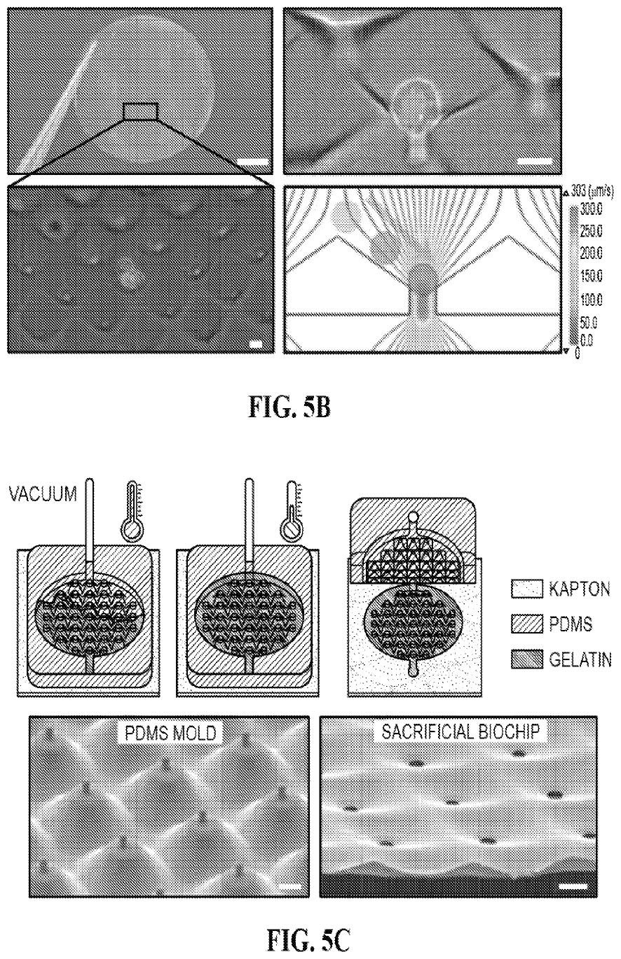

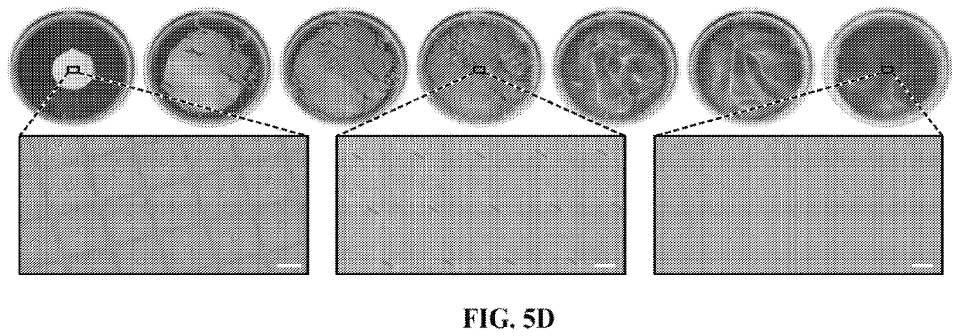

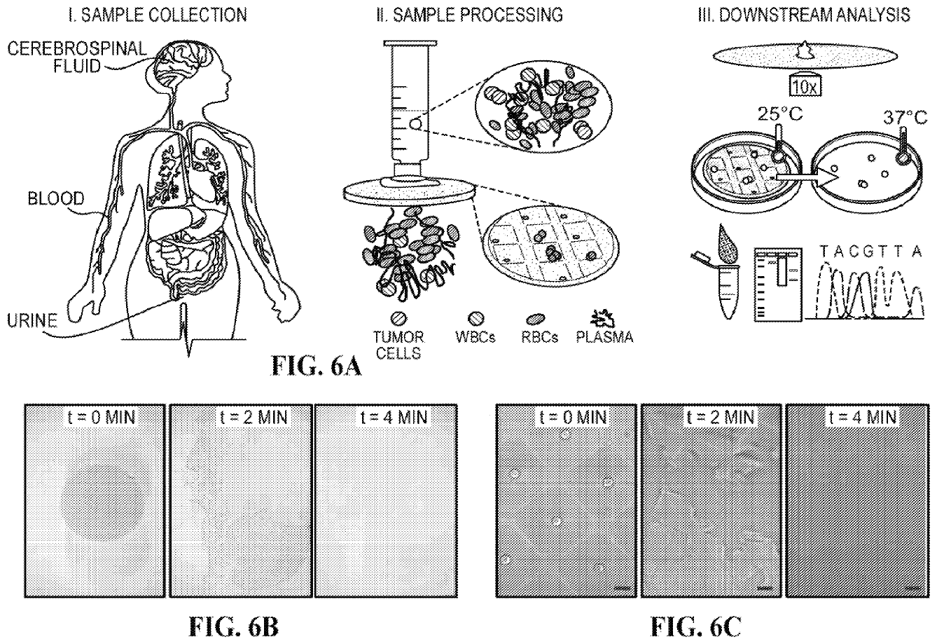

The sacrificial device (also referred to as a “sacrificial biochip”) can capture tumor cells from a patient sample before it discreetly disintegrates when it is deemed idle for the subsequent investigation. To achieve this transitory response, the sacrificial device is manufactured out of gelatin, a thermo-responsive hydrogel that remains solid at room temperature and liquifies at physiological temperatures (FIG. 5A). By combining the features of microfiltration-based systems and the material properties of gelatin, the sacrificial device facilitates isolation of viable tumor cells from various liquid biopsy sources, imaging of isolated cells using fluorescence microscopy, and culturing of the enriched cells following the device vanishing process. Furthermore, the biocompatible nature of gelatin can make the sacrificial device, whether in its solid or liquefied state, suitable for a variety of cytologic and molecular assays including fluorescence microscopy, tissue culture or polymerase chain reaction (PCR) for direct integration of isolated cells with the downstream analyses without any need for staining, scanning and micromanipulation (FIG. 6A).

Unlike conventional filtration membranes, the sacrificial device disappears after enrichment processes, which can provide straightforward integration of isolated cells with the downstream analyses (FIG. 6B). The vanishment is initiated, in this embodiment, by increasing the temperature of the medium to the physiological rates (37° C.) at which the whole device fully disappears in minutes (FIG. 6C). The vanishing mechanism makes the isolated cells readily available for functional analyses including in vitro cell culture and drug testing as well as for molecular analyses without any need for additional processing steps.

An exemplary sacrificial device was designed in the form of a membrane filter to capture tumor cells based on their size difference from normal cells. An ~8 μm-diameter pore size was targeted based on previous filtration studies [34], [35], [36] and the pores were arrayed at a ~55 μm pitch to achieve ~160,000 pores over a filtration area of ~490 mm². The surface of the membrane filter was specifically designed to have a topography with slanted sidewalls (1) funneling the cells from all directions directly to pores and (2) minimizing stagnant flow regions near the surface compared to a conventional membrane filter to minimize non-specific adhesion of cells (FIG. 5B).

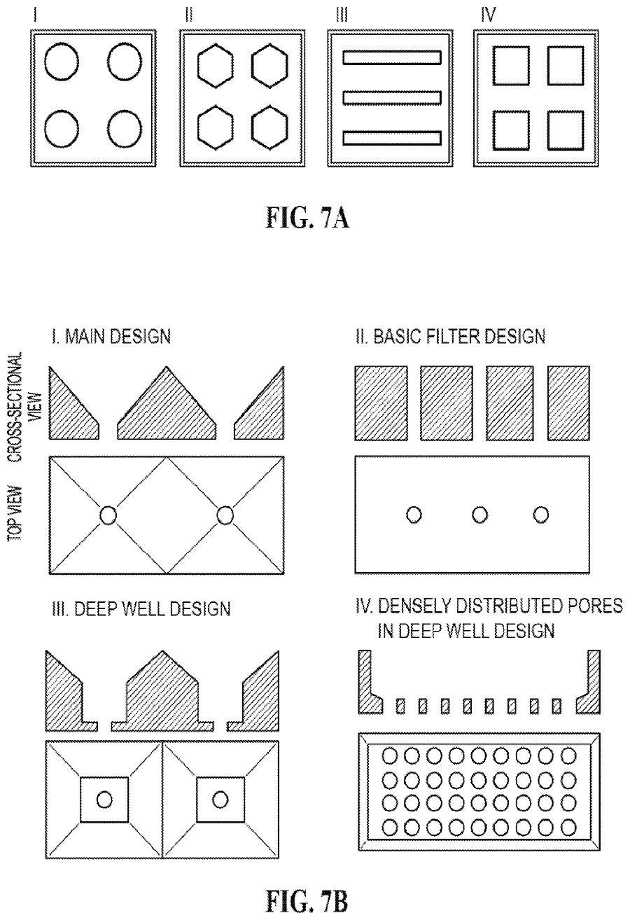

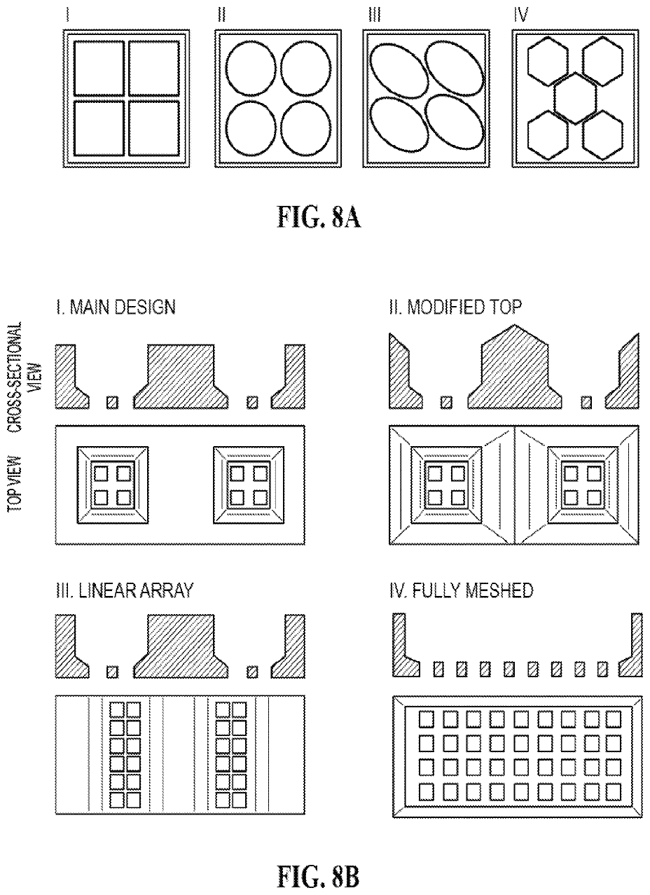

The pore size, design pitch, and pore shape can be changed according to the size and shape of the particle of interest, as shown in FIG. 7A (but not limited by the examples in FIG. 7A). Besides pore size, the device structure can also be changed as shown in FIG. 7B (not limited by the examples in FIG. 7B). Any combination of structural design and shape/size of pores would allow the device to be suitable for various applications. The developed vanishing device can also be used for isolating clustered particles by employing different designs that utilize support meshes to form a dynamic force balance needed for stable equilibrium of clustered particles. The pore shape and size can be arranged according to the particles of interest as illustrated in FIG. 8 (but not limited by the examples in FIG. 8).

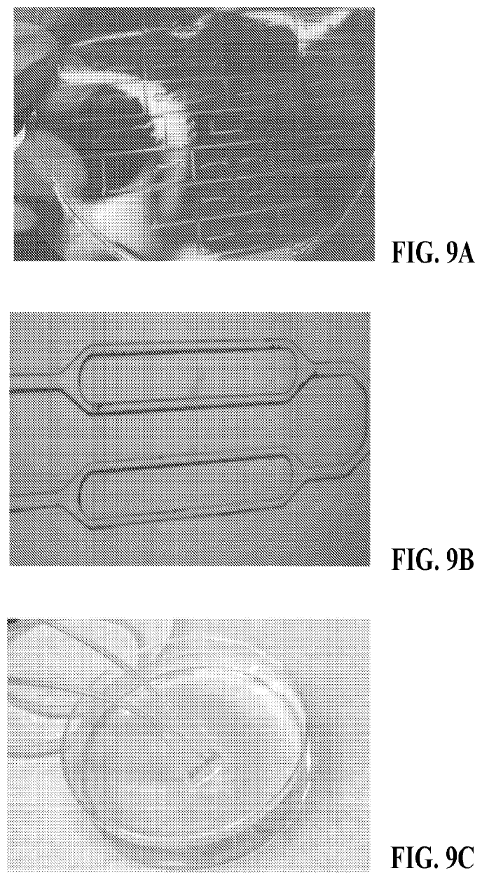

Different concentrations of gelatin solutions can readily be patterned on a previously patterned substrate using soft-lithography techniques, e.g., conventional soft-lithography techniques (FIGS. 9A-9B). It is contemplated that the low cost, reproducibility, mass production capability, and vanishing property of microfluidic devices (FIG. 9C) render the vanishing device and its fabrication processes applicable to a wide range of microfluidic applications.

### Exemplary Method of Fabrication

Tumor cell enrichment technologies have been realized using silicon micromachining and conventional soft-lithography based fabrication methods. While silicon micromachining can be used for patterning complex 3D structures, its dependency on expensive cleanroom equipment and time-consuming process requirements limits its use in such single-use, disposable devices. On the other hand, soft lithography-based fabrication methods allow inexpensive and simple fabrication of devices in a laboratory environment without a need for complex equipment. However, devices realized by soft-lithography techniques are constrained to have relatively simpler structures, limiting design variations and device functionality.

Sacrificial biochips can be realized by a fabrication process developed to (1) mold gelatin with microscale precision, (2) reinforce it to provide mechanical rigidity while retaining its solubility, and (3) compensate for swelling-induced changes in wet versus dry device geometry. Through this microfabrication strategy, a membrane filter can be created with a surface topography optimized to minimize stagnant flow formation and actively guide the cells to uniformly spaced pores through slanted sidewalls, all leading to decreased retention of contaminating cells. It should also be noted that, while sacrificial devices fabricated as membrane filters are exemplified, the developed microfabrication process can be utilized to create different cell manipulation/capture structures and enclosed microfluidic channels and can potentially be combined with functionalization chemistries to increase specificity by embedding antibodies in the structure without incurring the challenges in releasing the cells immobilized on the device.

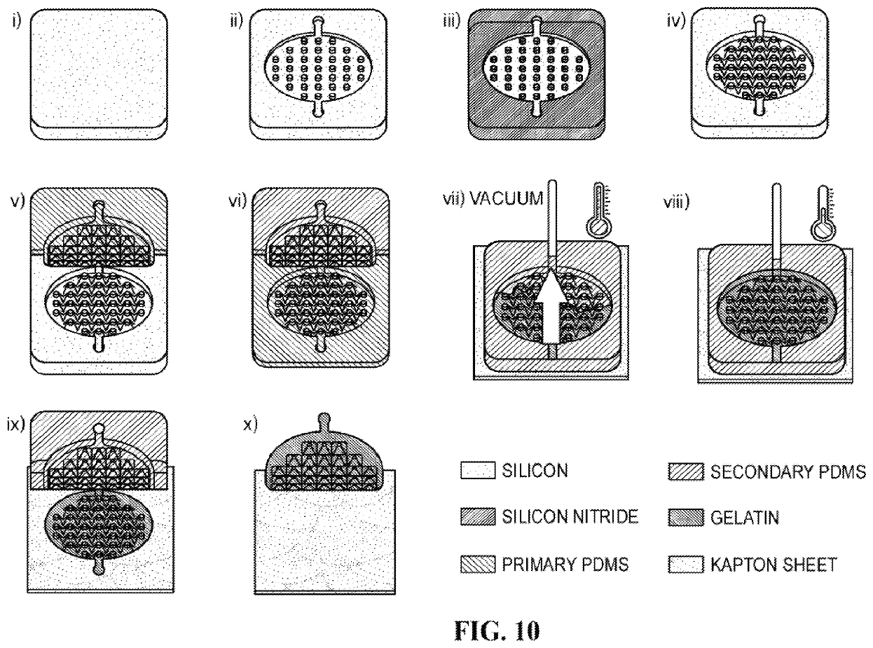

To shape gelatin into the desired device geometry, a fabrication method can utilize silicon micromachining, soft lithography, and micro-molding (FIG. 5C). First, a negative mold can be created in silicon through a multi-step process that involves optical lithography and thin film deposition with anisotropic etching (FIG. 10). The silicon mold geometry can then be transferred into polydimethylsiloxane (PDMS) and replicated using soft lithography to create an elastic, deformable mold. The PDMS mold can later be attached to a Kapton sheet, and the enclosed channels filled with a pre-heated gelatin solution on a thermoelectric surface heated beyond the melting temperature of gelatin to ensure against premature solidification. Infused gelatin can then be first solidified by lowering the surface temperature, removed from the PDMS mold, and then dried at room temperature before it can be delaminated from the Kapton sheet.

To ensure the microfabricated sacrificial device can withstand the stresses during handling and sample processing, its mechanical strength can be increased by physical crosslinking. Specifically, the gelatin devices can be incubated in Dulbecco's phosphate-buffered saline (DPBS) buffer containing divalent Mg²⁺ and Ca²⁺ metal ions that form ionic bonds with the carboxylic group of the polypeptide chains of gelatin, thereby making strong physical ionic crosslinking in the gel network, and thus increasing the mechanical strength and viscoelasticity of the sacrificial device [37]. This process can allow high elasticity, good mechanical strength, and low vulnerability of gelatin structures in aqueous solution. Final devices can withstand sample flow, successfully isolating cells from suspensions while still retaining thermo-responsive characteristics, with the whole device disappearing in minutes when temperature is increased to 37° C. (FIG. 5D).

Combination of the fabrication methodology with the nature of the gelatin hydrogel formation process offers various applications to design devices using hybrid materials for other applications:

Near infrared light sensitive filters: The designed amount of gold nanorods can be mixed with gelatin material to fabricate a hybrid infrared (NIR) light sensitive and thermo-responsive device for highly efficient capture and specific local-release of target particles with significant purity level by a combination of the photothermal effect of gold nanorods and vanishing properties of the gelatin filter. In addition, the high binding affinity of Au nanosurface to thiol groups would offer great opportunity to functionalize the device without sacrificing the vanishing property of the material. As a result, this dual-functional hybrid device may show great performance in the capture and local-release of target particles with high specificity and selectivity, which promises great potential applications for enrichment, retrieval, and analysis of target particles.

Thermally resistant devices: Besides the thermo-responsive property of the gelatin material, gelatin filters can also be designed as thermally resistant devices by soaking in 5% glutaraldehyde solution (in any other crosslinker solution) following the fabrication of the devices. Moreover, active aldehyde surface can easily be functionalized using designed conjugation chemistry for target specific applications such as antibody-specific capture of cells.

Time-adjustable vanishing devices: Metal 2+ ions such as Ca²⁺ and Mg²⁺ manipulates the vanishing time of gelatin devices. As a result, arranging metal 2+ ions concentration of device wetting solutions would offer opportunities to design time adjustable/programmable vanishing devices. Besides using ions, the addition of other solutions/materials into gelatin would also change the structural rigidity and resilience to vanishing.

Enrichment and Molecular Analysis of Clustered Particles: The sacrificial devices described herein can also be used for isolating clustered particles by employing different designs that utilize support meshes to form a dynamic force balance needed for a stable equilibrium of clustered particles. The pore shape and size can be arranged according to the particles of interest.

Isolation and Analysis of Pathogens: Besides isolation and downstream analysis of tumor cells, by employing proper changes in design and pore size, the developed technology can be utilized for enrichment and molecular analysis of bacteria and viruses in air and various liquids. The ability to concentrate pathogens that are present in extremely low concentrations would pave the way for reliable and efficient testing of samples.

Methods

### Micromanufacturing of Sacrificial Biochip

First, the silicon master-mold was patterned using conventional microfabrication techniques. A 4-inch diameter (100) silicon wafer was coated with the SC1813 photoresist (Shipley, Marlborough, MA) and patterned using photolithography, which was used as a mask for the subsequent etch process. Following the photolithography, the silicon was etched to a depth of 8 μm using deep reactive ion etching (DRIE), which formed the circular pillars. The resultant structure was then coated with a thin layer of silicon nitride using an LPCVD furnace. The silicon nitride layer was patterned by reactive ion etching (RIE), where SPR 220-7.0 positive photoresist (Shipley, Marlborough, MA) was used as a mask. Following the nitride patterning, the silicon was anisotropically etched to a depth of 40 μm in a 25% TMAH+1% Triton-X solution at 80° C. to form the slanted walls. The fabricated silicon master-mold was then coated with trichloro(octyl)silane under vacuum conditions for 8 hours prior to polydimethylsiloxane (PDMS) casting.

After silicon micromachining, the negative pattern of the silicon master-mold was transferred to a PDMS layer (primary PDMS) using the soft-lithography method, and the surface of the primary PDMS layer was activated using oxygen plasma and coated with trichloro(octyl)silane for 8 hours. Then, the primary PDMS layer was used as a mold for replicating the secondary PDMS layer, which had the inverse pattern of the device design that is intended to be fabricated. Following the PDMS-to-PDMS molding step, the secondary PDMS was placed on a Kapton sheet (McMaster-Carr, Cat No: 2271K41) and put on top of a thermoelectric heater/cooler set at 42° C., which prevents the preheated gelatin (Sigma Aldrich, Cat No: G1890 and Thermo-Fisher, Cat No: G13186) from solidifying during mold filling. The enclosed PDMS-Kapton sheet stack was filled with 25% gelatin solution under a 100 mbar vacuum. Once the mold was fully filled, the thermoelectric heater/cooler was set to 8° C., and the gelatin was solidified for 30 min at the set temperature. After the solidification, the PDMS layer was removed from the gelatin filter/Kapton sheet stack. The fabrication process was followed by delaminating the patterned gelatin device from the Kapton sheet after a 10-minute drying process at room temperature.

Gelatin-based Bioinks for Extrusion-Based 3D-Bioprinting: In some embodiments, the exemplary vanishing device can be fabricated in part using additive manufacturing operations. Gelatin can indeed be used as a bioink for extrusion-based 3D bioprinting. The process involves extruding gelatin through a heated nozzle, which brings it close to its melting point. The gelatin is then deposited onto a cooled stage where it undergoes a gelation process, transitioning from a liquid or viscous state to a solid state.

The gel formation mechanism should be considered 3D bioprinting using gelatin as a bioink. The gelatin solution must have a suitable viscosity or viscoelasticity to facilitate the initial extrusion, but it should quickly solidify and become self-supporting after deposition to allow for the addition of additional layers.

Temperature control during the bioprinting process should be considered to prevent premature gelation of the gelatin solution while it is still inside the printer. If gelation occurs too early, it can clog the nozzle and disrupt the printing process. Therefore, the printer's design and temperature control system should be optimized to maintain the gelatin solution in a printable state until it reaches the cooled stage.

To improve the printability and mechanical properties of gelatin as a bioink, physical crosslinking methods can be employed. One such method involves using different metal ions, typically divalent cations (2+ ions), to induce crosslinking of the gelatin molecules. These metal ions form coordination complexes with the gelatin chains, creating physical crosslinks that enhance the structural integrity and stability of the printed constructs. The choice of metal ions and the concentration of these ions can be optimized to achieve the desired printability and mechanical properties of the 3D-printed gelatin structures.

Overall, gelatin-based bioinks for extrusion-based 3D bioprinting offer versatility and potential in tissue engineering and regenerative medicine applications. Through careful control of gelation and the use of crosslinking agents, gelatin bioinks can be optimized for specific printing requirements and to mimic the native properties of different tissues.

### Sample Collection

Blood samples from consenting healthy donors were collected, along with blood samples from prostate cancer patients and an ovarian patient and a CSF sample from a medulloblastoma patient. The blood samples were collected in EDTA tubes (BD Vacutainer), and the CSF sample was collected in a CSF collection tube and processed within 4 h of sample withdrawal. To prevent sedimentation, tubes were placed on a rocker until use.

### Sample Processing

The devices were placed inside the filter holders and filled with 1× PBS. Prior to use, the device/filter holder assembly was incubated with 3% bovine serum albumin (BSA) for at least 1 h to minimize non-specific cell adhesion on the surfaces. Then, the BSA was washed away with 1× PBS before the introduction of the samples. The samples were run through the devices using a syringe pump (Harvard Apparatus Infuse/Withdraw PHD Ultra) under withdrawal mode at a 10 mL/h flow rate. Then, the devices were washed using 1× PBS solution for 1 h before the immunofluorescence staining or molecular and functional analyses.

### Cell Culture and Preparation

As model biological samples, LNCaP (ATCC-CRL-1740; Manassas, VA), ONS-76 (obtained from Dr. Tobey MacDonald, Emory University), and T24 (ATCC-HTB-4; Manassas, VA) cell lines were used. The cell lines were cultured in RPMI-1640 (LNCaP and ONS-76) or McCoy's 5A (T24) medium containing 10% fetal bovine serum (FBS) (Seradigm, Radnor, PA) in 5% CO₂ atmosphere at 37° C. Once they reached 80% confluence, cells were detached from the culture flask using 0.25% trypsin (Gibco) for 2 minutes. Subsequently, cells were pelleted, the supernatant was removed, and the cells were resuspended in 1× PBS solution by gentle pipetting.

For the characterization experiments, LNCaP cells were spiked into blood samples drawn from healthy donors, T24 cells were spiked into urine samples of healthy donors, and ONS-76 cells were spiked into aCSF samples prepared by mixing 124 mM NaCl, 2.5 mM KCl, 2.0 mM MgSO₄, 1.25 mM KH₂PO₄, 26 mM NaHCO₃, 10 mM glucose, 4 mM sucrose, and 2.5 mM CaCl₂ in DI water.

### Measurement of Device Capture Efficiency

The capture efficiency tests were performed by spiking pre-labeled LNCaP human prostate tumor cells into unprocessed whole blood samples drawn from healthy donors. The tumor cells were first fluorescently labeled using an orange CMRA cell tracker (Invitrogen), and the nuclei of cells were labeled by incubating the cells in a 4 mL Hoechst 33342 dye (Thermo Fisher, Cat No: H3570) in 1× PBS (1:1000) for 20 min in 5% CO₂ atmosphere at 37° C. After washing off the staining solutions, the cells were detached and resuspended in 1× PBS. The cell concentration was calculated using a Nageotte chamber prior to the experiments. The experiments were performed by spiking fluorescently labeled tumor cells into unprocessed whole blood samples at a final concentration of 1,000 cells/mL. The blood samples were processed and subsequently washed with 1× PBS using a syringe pump (Harvard Apparatus Infuse/Withdraw PHD Ultra) under withdrawal mode. Lastly, the spiked and captured populations were compared, and the device capture efficiency was calculated by counting the cells using a fluorescence microscope.

### Measurement of Cell Viability

To determine the viability of cells, live/dead assay (ab115347, Abcam, Cambridge, MA) was performed according to the manufacturer's instructions. The assay was mixed at 5× concentration (1:200) with the 1× PBS solution containing the control and released cells. Following the incubation in dark environment for 15 min, the samples were imaged with an inverted fluorescence microscope (Eclipse Ti, Nikon, Melville, NY), where the live and dead cells were observed in green (FITC) and red (TexasRed) channels, respectively.

### Measurement of Non-Specific Binding

To compare the non-specific protein binding of gelatin-based device with polycarbonate and polyester membrane filters, the filters were incubated with Cy-5 conjugated IgG protein solution (Invitrogen, Cat No: A11357) for an hour at room temperature. Following the incubation, the filters were washed with 1× PBS to remove excess unbound proteins. Filters were then imaged under a fluorescence microscope using Cy-5 channel to get the mean fluorescence signal levels. After subtracting the baseline fluorescence levels (autofluorescence) of each filter, the signal values corresponding to the non-specific protein binding were obtained.

### SEM Sample Preparation and Imaging

The captured cells were first fixed in 2.5% glutaraldehyde diluted in 0.1 M sodium cacodylate. After fixation, cells were dehydrated in 50%, 70%, 80%, and 95% ethanol solutions in water and 100% ethanol successively for 15 min in each. The sample was then dried at room temperature overnight. The device and captured cells were coated with Pt/Au using a sputtering system and imaged using a Hitachi SU8230 scanning electron microscope.

### Immunofluorescence Staining of Isolated Tumor Cells

After processing the samples, the captured cells were subjected to either live or fixed staining protocol. For live staining, isolated LNCaP prostate tumor cells were stained with Alexa 488-conjugated antibodies against EpCAM (Cell Signaling Technology, Cat No: 5198S), prostate-specific membrane antigen (PSMA) (BioLegend, Cat No: 342506, Clone: LNI-17), and the contaminating WBCs were stained with PE-CD45 (TRITC) (BioLegend, Cat No: 368510, Clone: 2D1) by incubating for an hour at room temperature. The device with captured cells was washed with 1× PBS prior to imaging. For fixed staining, isolated cells were first fixed with methanol for 10 min and subsequently permeabilized with 1% Triton-X (Sigma-Aldrich, St. Louis, MO) in PBS for 10 min. Prior to immunofluorescence staining, the device/filter holder assembly was incubated with a blocking buffer containing 2% goat serum and 3% BSA for 30 min. For prostate samples Cytokeratin 8/18 (Invitrogen, Cat No: MA5-32118, Lot No: UI2852552, Clone: SU0338), Vimentin (Invitrogen, Cat No: MA5-14564, Lot No: VF3005193, Clone: SP20), PSA/KLK3 (Cell Signaling Technology, Cat No: 5365S, Lot No: 4, Clone: D6B1), EpCAM (Invitrogen, Cat No: MA5-29246, Lot No: UJ2852411, Clone: 28), and Anti-CD45 (BD Biosciences, Cat No: 555480, Lot No: 8043547, Clone: HI30), for ovarian samples, Cytokeratin 7 (Invitrogen, Cat No: MA5-32173, Clone: ST50-05, (1:400)), Cytokeratin 8/18 (Invitrogen, Cat No: MA5-32118, Clone: SU0338, (1:400)), Vimentin (Invitrogen, Cat No: MA5-14564, Clone: SP20, (1:1000)) and Anti-CD45 (BD Biosciences, Cat No: 555480, Clone: HI30, (1:500)) and for medulloblastoma samples, Synaptophysin (BioLegend, Cat No: 837104), NCAM (Thermo Fisher, Cat No: MA5-11563), B7-H3 (Thermo Fisher, Cat No: MA5-29102), and Anti-CD45 (Cell Signaling Technology, Cat No: 13917S) primary antibody cocktail was introduced and incubated overnight. The excess antibodies were washed away with 1× PBS. Then, the matching secondary antibodies Alexa Fluor 488 (Invitrogen, Cat No: A-11008, Lot No: 1981125), Alexa Fluor 594 (Invitrogen, Cat No: A21125, Lot No: 2126810) for prostate and ovarian samples, and Alexa Fluor 488 (Thermo Fisher, Cat No: A-10680), Alexa Fluor 594 (Thermo Fisher, Cat No: A-11037) for medulloblastoma samples were run through the device, incubated for 1 h, and washed away with 1× PBS. The nuclei of cells were stained with 4′,6-diamidino-2-phenylindole (DAPI) (Invitrogen, Cat No: D1306) for 10 min, and the devices were washed with 1× PBS.

### Molecular Analysis of Isolated Cells

The captured cells were lysed, and mRNA of cells was isolated using Dynabeads oligo(dT)25 beads included in the ProstateCancerDetect kit. Subsequently, reverse transcription was performed using a Sensiscript RT kit (Qiagen, Hilden, Germany) and the cDNA was then used as a template in a multiplex PCR using the primer mix supplied from the kit manufacturer for the markers, including PSMA, PSA, EGFR, and Actin. For detecting androgen receptors, an AR-Detect kit (Qiagen, Hilden, Germany) was used as suggested by the manufacturer (Qiagen, Hilden, Germany). Visualization and quantification of transcripts were performed using an Agilent Bioanalyzer 2100 (Agilent, Böblingen, Germany). The results were considered to be positive if the fragment concentration of at least one of the markers was greater than 0.1 ng/μL for ProstateCancerDetect, and greater than 0.15 ng/μL for AR-Detect kits as suggested by the manufacturer.

### Structural and Chemical Characterization of Sacrificial Biochip

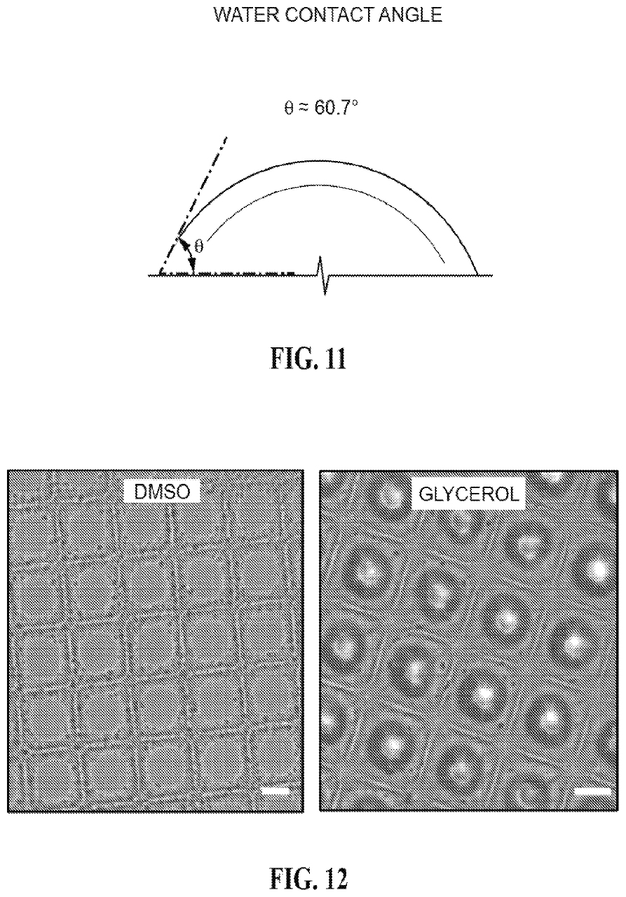

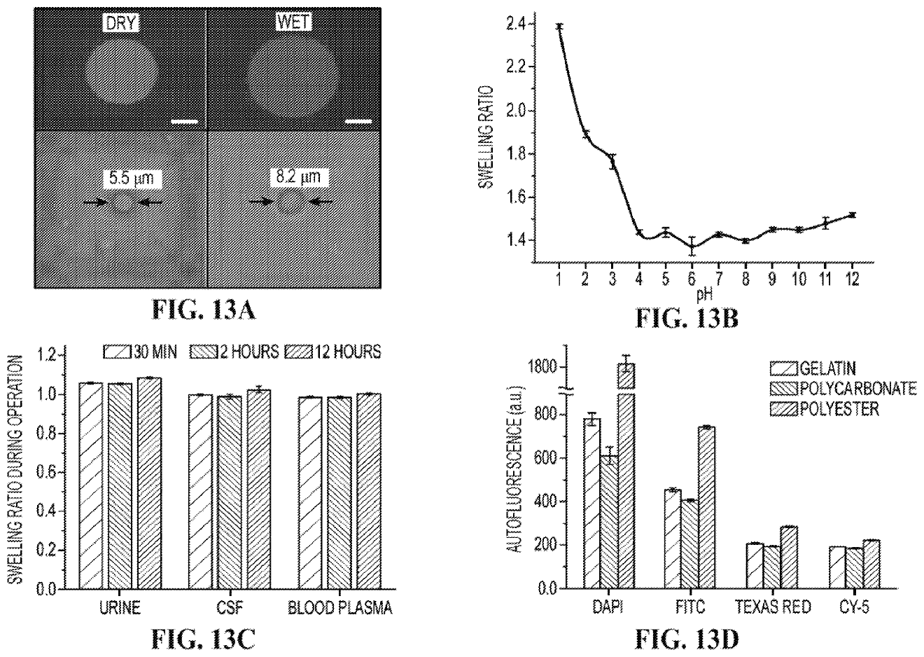

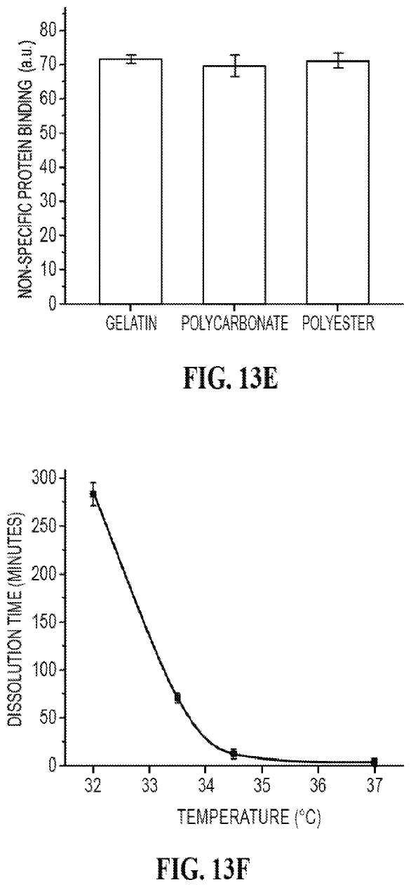

A study was conducted that investigated the compatibility of the sacrificial biochip with different liquid environments. The biochip was found to be hydrophilic with a water contact angle of <90° (FIG. 11), which made it effortlessly wettable in all liquids. It was observed in the study that the sacrificial biochip retained its structure in a variety of commonly used solvents (Table 1). Besides deionized (DI) water and phosphate-buffered saline (PBS), the sacrificial biochip was compatible with mildly polar organic solvents, such as primary alcohols and acetone, in which gelatin is insoluble [40]. Notably, the sacrificial biochip was dissolved in more polar organic solvents, including 100% glycerol and dimethyl sulfoxide (DMSO), while retaining its structure in diluted (10%) solutions of both solvents typically employed for cell freezing (FIG. 12). Next, the swelling ratio of the sacrificial biochip was measured in different liquids. While the swelling ratio of the device in 1× PBS, a commonly employed buffer in cell-based applications, was ~50.4%, virtually no swelling was observed in methanol, ethanol, isopropyl alcohol, and acetone. The effects of swelling on the device geometry were also characterized by measuring pore sizes before and after immersion in different liquids (Table 1). These results allowed compensation for swelling-induced changes in device geometry during microfabrication, i.e., the pores were fabricated with a diameter of 5.5 μm to achieve a final pore diameter of ~8 μm in immersion for 1× PBS (FIG. 13A).

Next, the effect of ambient pH changes on the sacrificial biochip was investigated. The study found that the biochip retained its structure under a broad pH range from 1 to 12, only dissolving in highly acidic (pH<1) or highly basic (pH>2) solutions (FIG. 13B). Swelling ratios of the sacrificial biochips were particularly sensitive to the changes in the pH between 1 to 4, while a more stable response for the pH levels 4-10 was observed, which pertain to most of the bodily fluids of interest for liquid biopsy (e.g., blood pH range 7.3-7.5, urine pH range 4.8-8.4, CSF pH range 7.3-7.5) [41]. In fact, incubating the sacrificial biochip in blood plasma, artificial CSF (aCSF), and urine for up to 12 hours resulted in minimal additional swelling (<7%), demonstrating the applicability of the technology for cell screening in different samples (FIG. 13C). It is contemplated that the chemistry of the example devices would be compatible for the biological and inorganic fluid environment having PH range of 4-10.

To test the compatibility of the sacrificial biochip with fluorescence imaging, autofluorescence of the device was measured, as well as commercial polycarbonate and polyester (PETE) membrane filters for comparison (FIG. 13D). Analyzing each platform for most commonly used fluorescence channels (i.e., DAPI (blue), FITC (green), TexasRed (red), and Cy-5 (violet)), the highest autofluorescence intensity was observed from the DAPI channel with autofluorescence decreasing with increasing emission wavelength for all platforms. The sacrificial biochip was found to show autofluorescence levels similar to the polycarbonate filter for all tested channels and was also less autofluorescent than the polyester filter. Taken together, these results demonstrated the feasibility of performing immunocytochemical analysis of cells captured on the device even when the device is present while imaging.

Furthermore, the study investigated the non-specific binding of proteins to the sacrificial biochip by benchmarking against commercial membrane filters that are specifically manufactured to minimize protein binding capacity [42]. To quantify non-specific protein binding, devices incubated with fluorophore-conjugated IgG protein were imaged via fluorescence microscopy (see Example 1: Methods). Comparing measured fluorescence emission levels, each corrected for autofluorescence (i.e., baseline) from the device, the study found that the sacrificial biochip, like the polycarbonate and polyester membrane filters, had a low protein binding capacity—a feature that is desirable for specific capture of target cells (FIG. 13E).

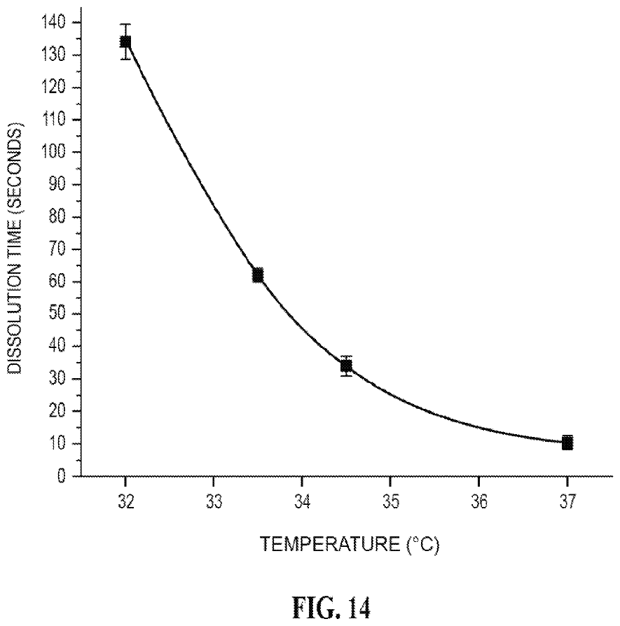

Finally, the study characterized the temperature-controlled dissolution of the sacrificial biochip by monitoring the process at different temperatures (FIG. 13F). The study found that the device can retain its structure for temperatures up to ~32° C. At 32° C., it took ~285 minutes for the device to completely disappear. Increasing the temperature to 33.5° C. reduced the required dissolution time to ~75 mins, while it took only ~4 mins for the complete dissolution of the device immersed in a 1× PBS solution pre-heated to 37° C. On the other hand, it was also observed in the study that devices not treated with divalent metal ions for mechanical strength dissolved much more rapidly for a given ambient temperature—an expected result due to the missing ionic crosslinking in the gel network (FIG. 14). Besides determining operable temperature ranges for the sacrificial biochip, these results also showed the feasibility of tuning the device dissolution time in different assays by modulating the ambient temperature.

### Isolation of Spiked Tumor Cells From Simulated Samples

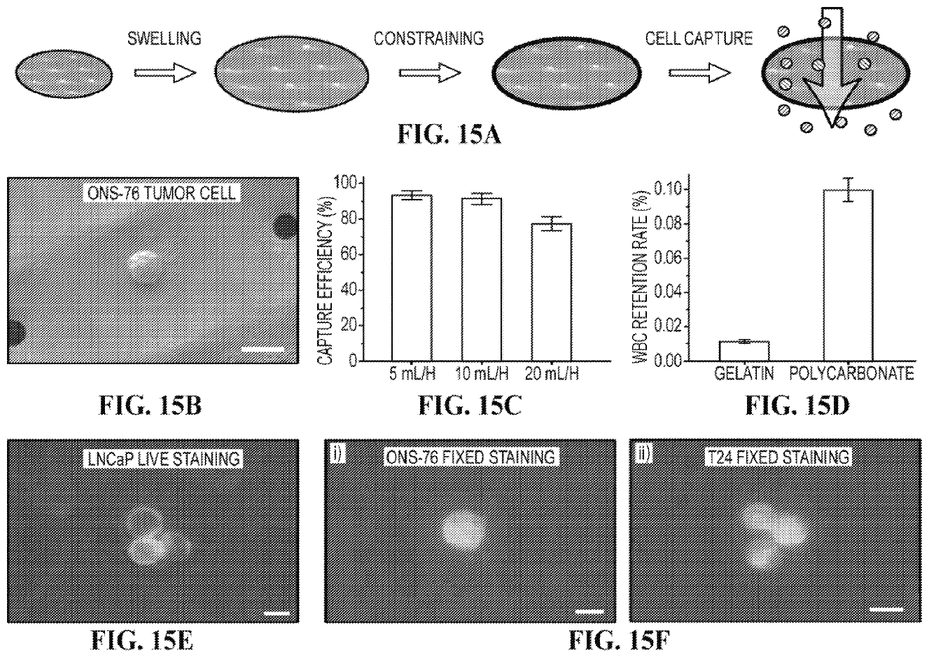

To install the sacrificial biochip in a standard aqueous filtration setup, the device was first pre-swelled in 1× DPBS, and then the swollen device was mechanically constrained at its rim by attaching an O-ring with a diameter matching the intended filter holder and samples processed to isolate target cells (FIG. 15A). To functionally test the sacrificial biochip for cell enrichment, control samples were first processed, which were prepared by spiking cultured human tumor cell lines into blood, urine and aCSF (FIG. 15B) (see Example 1: Methods). In these experiments, it was observed that the sacrificial biochip, placed in a commercial filter holder, successfully withstood the shear from the flow of whole blood, a dense and viscous liquid, and captured the spiked tumor cells. To quantify tumor cell capture efficiency, the number of cells retained on the biochip was compared with the number of spiked tumor cells, and the capture rate was calculated for different sample flow rates (FIG. 15C). The sacrificial biochip was able to isolate >90% of LNCaP human prostate tumor cells for sample flow rates up to 10 mL/h (~93.2% at 5 mL/h and ~91.3% at 10 mL/h), while the capture rate was reduced to ~77.3% at 20 mL/h. Based on the observed trade-off between the cell capture rate and the sample flow rate, the 10 mL/h flow rate was chosen as the optimum flow rate for the tumor cell isolation experiments.

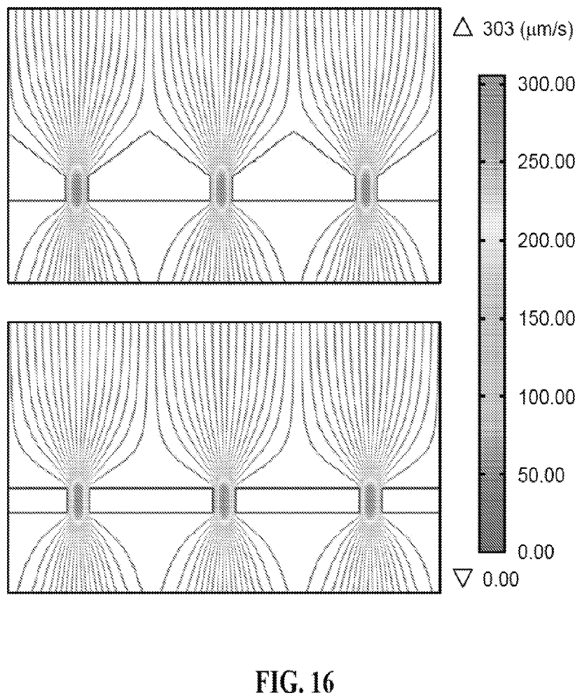

Next, the study investigated the retention of normal cells on the sacrificial biochip as those cells would produce artifacts in fluorescence imaging and molecular assays. Comparing the sacrificial biochip with a commercial track-etched polycarbonate membrane filter with a similar pore size, both processing matched 10 mL of whole blood samples at 10 mL/h, the study found an order of magnitude reduction in the retention of white blood cells on the sacrificial biochip (FIG. 15D). Given that a similar protein binding capacity was observed between the sacrificial biochip and polycarbonate filters (FIG. 13E), the observed purity enhancement in the biochip may be attributed to its unique surface topography that minimizes stagnant flow formation on the filter surface by funneling the flow to the pores through slanted sidewalls (FIG. 16).

To test the compatibility of the sacrificial biochip with immunocytochemistry workflows, the study attempted to immunostain the captured cells directly on the biochip following the enrichment process. First, membrane antigens of unfixed cells were stained, specifically LNCaP human cancer cells isolated from whole blood along with the contaminating WBCs found on the biochip, using fluorophore-conjugated anti-EpCAM and anti-CD45 (see Example 1: Methods). Subsequent imaging of the biochip with fluorescence microscopy showed both the tumor cells and WBCs were stained with specific markers against minimal background (FIG. 15E). Next, the study investigated immunostaining of fixed cells on different combinations of tumor cell types and bodily fluids, specifically spiked ONS-76 medulloblastoma tumor cells isolated from an aCSF sample (FIG. 15F (i)) and spiked T24 bladder tumor cells isolated from a urine sample (FIG. 15F (ii)). Tumor cells were fixed on the sacrificial biochip and then immunostained with antibodies against tumor-specific membrane and cytoplasm markers (NCAM [43], B7-H3 [44], Synaptophysin [45] for medulloblastoma tumor cells and Cytokeratin 8/18 [46] for bladder tumor cells). The nuclei of the permeabilized cells were also stained with 4′,6-diamidino-2-phenylindole (DAPI). Taken together, these results validated the utility of the sacrificial biochip for on-chip immunocytochemistry assays commonly used to positively identify enriched tumor cells in the presence of contaminating healthy cells.

### Analysis of Enriched Tumor Cells Post-Dissolution of Sacrificial Biochip

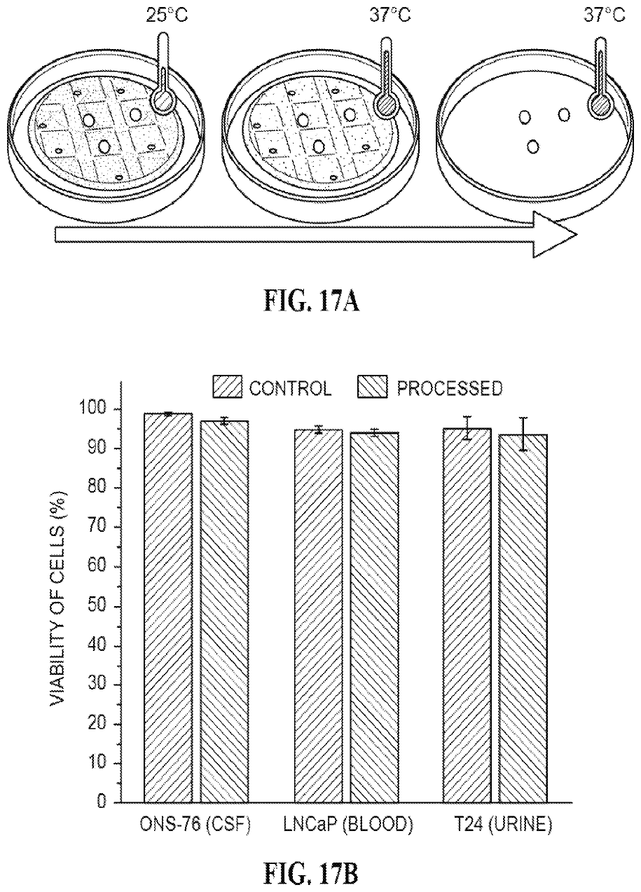

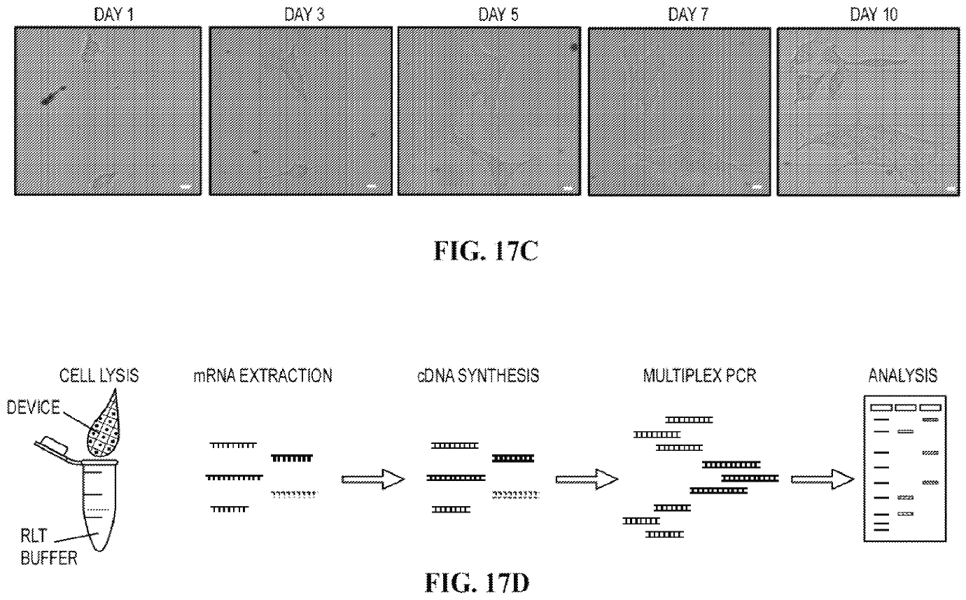

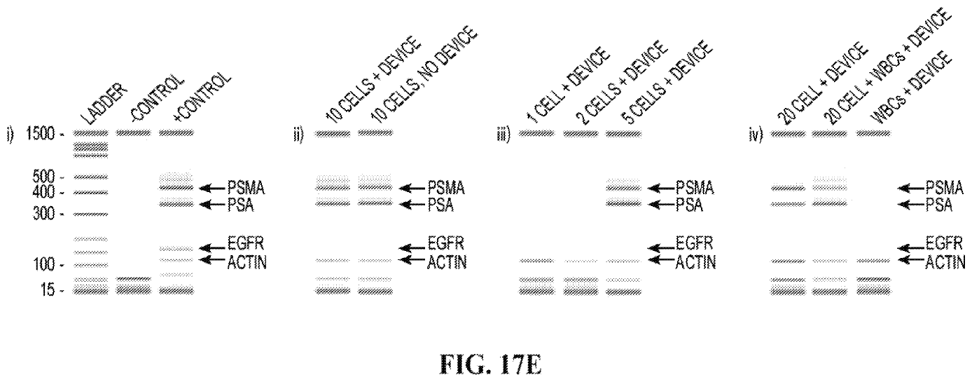

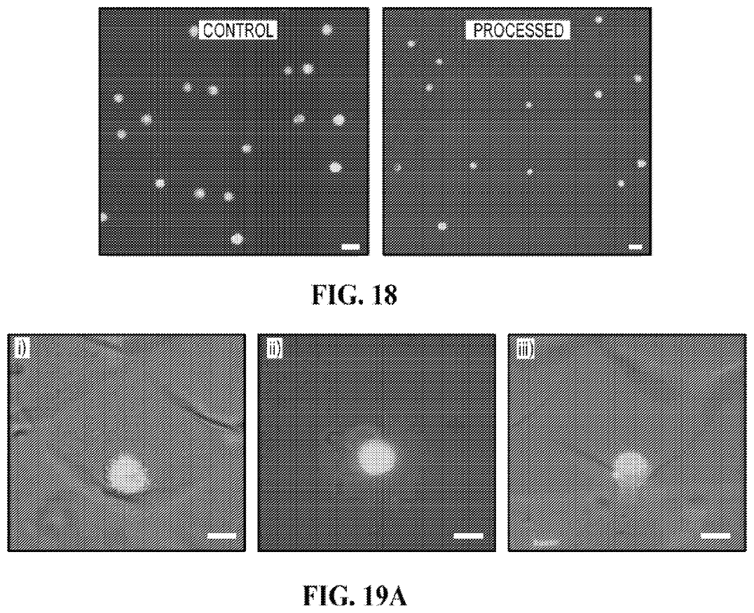

The study investigated viability of enriched tumor cells after the sacrificial biochip was dissolved by applying heat. In these experiments, microchips used to process samples were washed and directly placed in Petri dishes, where they were subjected to a 37° C.-ambience in an incubator (see Example 1: Methods, FIG. 17A). The study found that the viability of the released cells was similar to the control (unprocessed) populations for all of the tested samples, the LNCaP prostate cancer cells isolated from blood, ONS-76 medulloblastoma cancer cells isolated from aCSF, and T24 bladder cancer cells isolated from urine samples (FIG. 17B, FIG. 18). As another validation of cell viability, the study also attempted to culture released ONS-76 cells for a 10-day period and successfully observed expansion of cells (FIG. 17C). These results confirmed the non-cytotoxic nature of the developed technology as well as the advantages from its stress-free handling and release.

Next, the study integrated the enrichment assay with a molecular analysis workflow to analyze the RNA expression of isolated tumor cells. In this aspect of the study, the sacrificial biochip was folded and placed inside a PCR tube together with captured LNCaP prostate tumor cells. RLT buffer supplemented with 1% BME (see Example 1: Methods) in the PCR tube was observed to dissolve the sacrificial biochip in seconds along with lysing the cells on the device (FIG. 17D). Following the mRNA isolation and cDNA synthesis steps (see Example 1: Methods), multiplexed Reverse Transcript Polymerase Chain Reaction (RT-PCR) was performed using a commercial primer mix ProstateCancerDetect kit of AdnaTest® (Qiagen, Hilden, Germany). Prostate specific membrane antigen (PSMA), prostate specific antigen (PSA), and epithelial growth factor receptor (EGFR) were targeted for amplification as tumor-associated markers, while Actin was amplified as control. Samples, in which successful amplification of at least one tumor-associated target with a concentration >0.1 ng/μL along with the control was interpreted as tumor-positive as suggested by the manufacturer.

To characterize the developed molecular assay, the resultant concentration of amplicons associated with the targeted prostate cancer-specific genes was measured for different control samples via electrophoresis (see Example 1: Methods). First, the study investigated whether the presence of dissolved sacrificial biochip affected the assay by comparing the electropherogram from a pure population of 10 LNCaP cells and 10 LNCaP cells mixed with the sacrificial biochip and concluded that the microchip did not produce any noticeable effect on the quality of the resultant amplicons or on the process (FIG. 17E, panel (ii)). Second, the sensitivity of the assay for detecting rare tumor cells was tested by processing varying numbers of LNCaP prostate tumor cells mixed with a sacrificial biochip. While clearly visible PSMA and PSA bands were observed, which agree with published data [47], from as low as 5 cells, the same as the manufacturer-provided sensitivity for the primer mix, no signal was detected from 1 or 2 LNCaP cells (FIG. 17E, panel (iii)). Finally, the study evaluated the assay operation in the presence of contaminating cells in the sample. To simulate actual enriched tumor cell samples, which unavoidably contains host cells, spiked 5,000 WBCs were spiked into samples containing tumor cell and the sacrificial biochip (FIG. 17E, panel (iv)). The electropherogram from these samples (20 cells+WBC+device) resulted in the same prostate cancer-specific bands (PSMA and PSA) as pure samples, while showing extra dimmer bands due to increased background noise. In contrast, the control sample containing only 5,000 WBCs with the sacrificial biochip (WBC+device) was positive only for Actin, thereby validating the specificity of the results. Taken together, these results demonstrated the feasibility of natively integrating the enrichment of tumor cells using the sacrificial biochip with a molecular assay to create a cell-based liquid biopsy with specificity to expression of tumor-specific genes.

### Analysis of Patient Tumor Cells with Sacrificial Biochip

To demonstrate the clinical utility of the developed technology, the study processed blood samples of patients with metastatic prostate and ovarian cancer, and a CSF sample of medulloblastoma patients.

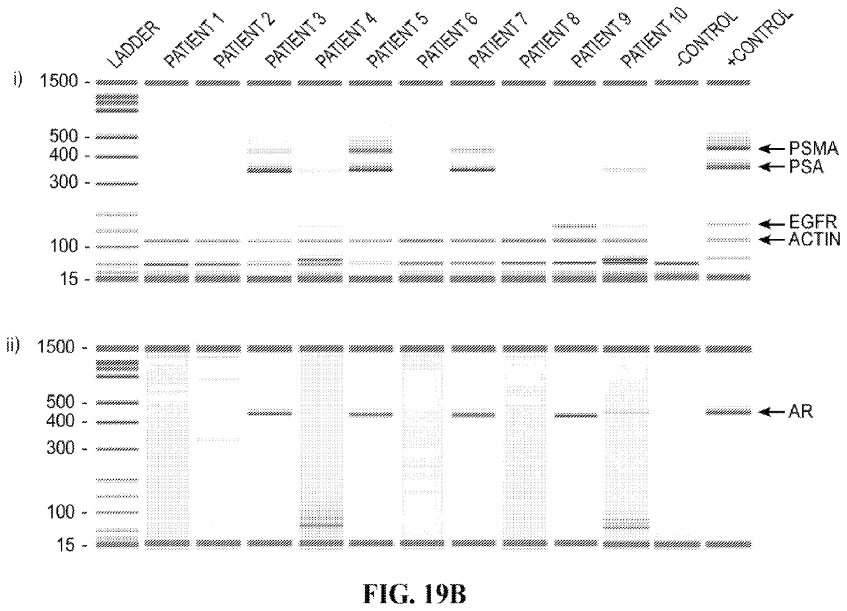

For molecular analysis, blood samples of prostate cancer patients were processed using the sacrificial biochips and isolated cells embedded within the sacrificial devices were directly subjected to PCR amplification (FIG. 19B). Following the cDNA synthesis (see Methods), multiplexed RT-PCR was performed on the samples for PSMA, PSA, EGFR and androgen receptor (AR) as tumor-associated markers, while Actin was targeted as an internal control. Among the cohort studied, all 10 samples were positive for internal control Actin, which showed the successful sample preparation and validity of the results. Tumor-associated markers were detected in 6/10 patients. Five patients were positive for AR expression (concentrations>0.15 ng/μL), among which three patients also showed positive bands for PSMA and PSA. Furthermore, among the six positive cases, patients 3, 4, and 5 were positive for EGFR, PSMA, and PSA, respectively. The variation in tumor-associated markers among the patients showed the interpatient heterogeneity of tumor cells.

The sacrificial biochip was used in practical and established cell enrichment and analysis workflows processing different types of samples. Due to its protein-based construction, the sacrificial devices were more compliant than their synthetic counterparts, yet their crosslinked polymer frame survived various bodily fluids and flow-induced shear forces. Their resilience against cell fixatives such as methanol and low autofluorescence for standard excitation and fluorescence emission channels enabled on-chip labeling and imaging of isolated cells. Furthermore, isolated cells could readily be transferred between assays as embedded within the device and remained viable after the dissolution of the device, as demonstrated by a short-term culture of isolated tumor cells.

Using sacrificial biochip, tumor cells were successfully isolated from liquid biopsy samples of cancer patients. Besides permitting conventional immunofluorescence imaging-based identification of isolated tumor cells, the whole device embedded with live tumor cells could directly be subjected to a commercial multiplex RT-PCR assay for detection of tumor-specific transcripts. In fact, expression of PSMA, PSA, EGFR, and AR from CTCs of prostate cancer patients could be detected directly from their unprocessed whole blood samples without the need for immunostaining, imaging, or micromanipulating individual cells. Therefore, the combined assay not only provided digital information on the presence or absence of viable CTCs in the blood sample with molecular specificity but also revealed inter-patient heterogeneity in the expression of biomarker transcripts within the studied cohort. Taken together, the inconspicuous nature of the sacrificial biochips circumvents challenges in integrating enrichment and analysis in cell-based assays and facilitates the development of practical-to-use, sample-to-answer tests to be utilized in research or routine clinical practice.

### Discussion

Here, a sacrificial microdevice is introduced, which is manufactured out of hydrogel and retains its structure only when needed, i.e., while screening a sample to isolate tumor cells, and subsequently dissolves to leave behind intact tumor cells for analysis. Unlike the conventional approach of releasing enriched tumor cells from an enrichment device, which compromises recovery rate and/or cell viability, the enrichment device itself is released from the retained tumor cells without any damaging or lossy processes. By micromachining a tumor cell enrichment device completely out of an organic material, a thermo-responsive hydrogel, it is ensured that the enrichment apparatus only exists when needed, during sample screening, and vanishes on demand either on a glass slide or in a container such as a test tube, petri dish or cell culture flask depending on the assay.

Using this technology, tumor cells were isolated from different samples including clinical ones collected from patients with metastatic prostate and ovarian cancers as well as medulloblastoma, and successfully subjected isolated tumor cells to immunocytochemical, functional, and molecular assays. As a showcase for the utility of the developed technology in research or clinical workflows, an assay was also developed, where the RNA expression of tumor-specific genes in CTCs could be detected directly from whole blood samples with no cell micromanipulation. Inconspicuous technologies for liquid biopsy will enable artifact-free, comprehensive analysis of tumor cells by natively integrating with other cytology and molecular assays.

Isolation of tumor cells shed into body fluids, called liquid biopsy, is a non-invasive technique for the detection and molecular analysis of cancer. Although there are numerous technologies developed for the reliable isolation of tumor cells from body fluids, there are few technologies targeting an effective release of isolated cells for further molecular and functional analyses without compromising their viability. Here, the exemplary vanishing device of a study utilized the thermo-responsive properties of gelatin for use in a filtration-based cell enrichment method that facilitates the lossless and stress-free release of isolated cells by vanishing the device on demand for downstream analyses. The exemplary device was, among other things, used for isolation and molecular analysis of circulating tumor cells from blood samples of metastatic prostate cancer patients. The exemplary vanishing device provides convenience and ease of use and can bypass the need for staining, scanning, and micromanipulation of target tumor cells, thus opening new avenues for the utilization of isolated tumor cells in research and clinical settings.

While numerous devices and associated techniques have been developed for isolation of targeted materials from obtained samples, many are not suitable for use in the isolation or enrichment of more sensitive materials, such as cells or other biological materials, due to the sometimes-harsh conditions required for separation of such materials from the associated devices. Often, maintenance of the integrity of the material is necessary for later analyses to be performed. In some instances, it is not possible to separate such targeted materials from devices or materials used in prior separation steps, which may limit the types of analytical techniques that can later be used in the characterization of the isolated materials. Thus, there is a clear need for new devices and methods which can allow clean separation of these types of materials while avoiding the above identified issues.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

The following patents, application, and publication as listed below and throughout this document are hereby incorporated by reference in their entirety herein.

### REFERENCES

[1] C. Alix-Panabie\grave{r}es, K. Pantel, Clin Chem 2013, 59, 110.
[2] N. Reimers, K. Pantel, Clinical Chemistry and Laboratory Medicine 2019, 57, 312.
[3] D. A. Haber, V. E. Velculescu, Cancer Discovery 2014, 4, 650.
[4] A. A. Ayantunde, S. L. Parsons, Annals of Oncology 2007, 18, 945.
[5] R. N. Garrison, L. D. Kaelin, L. S. Heuser, R. H. Galloway, Annals of Surgery 1986, 203, 644.
[6] M. Boya, T. Ozkaya-Ahmadov, B. E. Swain, C. H. Chu, N. Asmare, O. Civelekoglu, R. Liu, D. Lee, S. Tobia, S. Biliya, L. D. McDonald, B. Nazha, O. Kucuk, M. G. Sanda, B. B. Benigno, C. S. Moreno, M. A. Bilen, J. F. McDonald, A. F. Sarioglu, Nat Commun. 2022, 13, 1.
[7] M. T. J. V. Bussel, D. Pluim, B. M. Kerklaan, M. Bol, K. Sikorska, D. T. C. Linders, D. V. D. Broek, J. H. Beijnen, J. H. M. Schellens, D. Brandsma, Neurology, 2020, 94, e521.
[8] A. S. Patel, J. E. Allen, D. T. Dicker, K. L. Peters, J. M. Sheehan, M. J. Glantz, W. S. El-Deiry, Oncotarget 2011, 2, 752.
[9] X. Lin, M. Fleisher, M. Rosenblum, O. Lin, A. Boire, S. Briggs, Y. Bensman, B. Hurtado, L. Shagabayeva, L. M. DeAngelis, K. S. Panageas, A. Omuro, E. I. Pentsova, Neuro Oncol. 2017, 19, 1248.
[10] A. Chen, G. Fu, Z. Xu, Y. Sun, X. Chen, K. S. Cheng, K. H. Neoh, Z. Tang, S. Chen, M. Liu, T. Huang, Y. Dai, Q. Wang, J. Jin, B. Jin, R. P. S. Han, Cancer Res. 2018, 78, 4073.
[11] I. Leotsakos, P. Dimopoulos, E. Gkioka, P. Msaouel, A. Nezos, K. G. Stravodimos, M. Koutsilieris, C. A. Constantinides, Anticancer Res. 2014, 34, 7415.
[12] Z. Wang, J. Chen, L. Yang, M. Cao, Y. Yu, R. Zhang, H. Quan, Q. Jiang, Y. Hua, W. Wei, P. Lu, J. Wu, Q. Shi, Anal Chem. 2020, 92, 16284.
[13] L. Liang, Y. Wang, S. Lu, M. Kong, Y. Lin, F. Cuzzucoli, P. Wang, S.Q. Wang, Anal Chim Acta 2018, 1044, 93.
[14] M. Poudineh, E. H. Sargent, K. Pantel, S. O. Kelley, Nature Biomedical Engineering 2018, 2, 72.
[15] C. Alix-Panabieres, K. Pantel, Nature Reviews Cancer 2014, 14, 623.
[16] L. Wu, X. Xu, B. Sharma, W. Wang, X. Qu, L. Zhu, H. Zhang, Y. Song, C. Yang, Small Methods 2019, 3, 1800544.
[17] X. Wei, K. Chen, Z. Wang, B. Huang, Y. Wang, M. Yu, W. Liu, S. S. Guo, X. Z. Zhao, Particle and Particle Systems Characterization 2019, 36, 1900076.
[18] S. Hou, L. Zhao, Q. Shen, J. Yu, C. Ng, X. Kong, D. Wu, M. Song, X. Shi, X. Xu, W. H. Qu Yang, R. He, X. Z. Zhao, T. Lee, F. C. Brunicardi, M. A. Garcia, A. Ribas, R. S. Lo, H. R. Tseng, Angewandte Chemie 2013, 52, 3379.
[19] J. F. Swennenhuis, A. G. J. Tibbe, M. Stevens, M. R. Katika, J. V. Dalum, H. D. Tong, C. J. M. V. Rijn, L. W. M. M. Terstappen, Lab on a Chip 2015, 15, 3039.
[20] S. Zheng, H. K. Lin, B. Lu, A. Williams, R. Datar, R. J. Cote, Y. C. Tai, Biomedical Microdevices 2011, 13, 203.
[21] S. W. Lv, Y. Liu, M. Xie, J. Wang, X. W. Yan, Z. Li, W. G. Dong, W. H. Huang, ACS Nano 2016, 10, 6201.
[22] Q. Huang, B. Cai, B. Chen, L. Rao, Z. He, R. He, F. Guo, L. Zhao, K. K. Kondamareddy, W. Liu, S. Guo, X. Z. Zhao, Adv Healthcare Mater. 2016, 5, 1554.
[23] S. Hou, H. Zhao, L. Zhao, Q. Shen, K. S. Wei, D. Y. Suh, Advanced Materials 2013, 25, 1547.
[24] Y. Xu, J. A. Phillips, J. Yan, Q. Li, Z. H. Fan, W. Tan, Anal Chem. 2009, 81, 7436.
[25] Y. J. Kim, Y. T. Kang, Y. H. Cho, Anal Chem. 2016, 88, 7938.
[26] D. L. Adams, P. Zhu, O. V. Makarova, S. S. Martin, M. Charpentier, S. Chumsri, S. Li, P. Amstutz, C. M. Tang, RSC Adv. 2014, 4, 4334.
[27] H. J. Lee, J. H. Oh, J. M. Oh, J. M. Park, J. G. Lee, M. S. Kim, Y. J. Kim, H. J. Kang, J. Jeong, S. il Kim, S. S. Lee, J. W. Choi, N. Huh, Angewandte Chemie—International Edition 2013, 52, 8337.
[28] S. W. Lv, J. Wang, M. Xie M, N. N. Lu, Z. Li, X. W. Yan, S. L. Cai, P. A. Zhang, W. G. Dong, W. H. Huang, Chem Sci. 2015, 6, 6432.
[29] N. N. Lu, M. Xie, J. Wang, S. W. Lv, J. S. Yi, W. G. Dong, W. H. Huang, ACS Appl Mater Interfaces 2015, 7, 8817.
[30] X. Yu, B. Wang, N. Zhang, C. Yin, H. Chen, L. Zhang, B. Cai, Z. He, L. Rao, W. Liu, F. B. Wang, S. S. Guo, X. Z. Zhao, ACS Appl Mater Interfaces 2015, 7, 24001.
[31] N. M. Karabacak, P. S. Spuhler, F. Fachin, E. J. Lim, V. Pai, E. Ozkumur, J. M. Martel, N. Kojic, K. Smith, P. Chen, J. Yang, H. Hwang, B. Morgan, J. Trautwein, T. A. Barber, S. L. Stott, S. Maheswaran, R. Kapur, D. A. Haber, M. Toner, Nat Protoc. 2014, 9, 694.
[32] S. H. Au, J. Edd, A. E. Stoddard, K. H. K. Wong, F. Fachin, S. Maheswaran, D. A. Haber, S. L. Stott, R. Kapur, M. Toner, Sci Rep. 2017, 7, 1.
[33] Z. Liu, Y. Huang, W. Liang, J. Bai, H. Feng, Z. Fang, G. Tian, Y. Zhu, H. Zhang, Y. Wang, A. Liu, Y. Chen, Lab on a Chip 2021, 21, 2881.
[34] M. D. Zhou, S. Hao, A. J. Williams, R. A. Harouaka, B. Schrand, S. Rawal, Z. Ao, R. Brenneman, E. Gilboa, B. Lu, S. Wang, J. Zhu, R. Datar, R. Cote, Y. C. Tai, S. Y. Zheng, Sci Rep. 2014, 4, 1.
[35] G. Vona, L. Estepa, C. Beroud, D. Damotte, F. Capron, B. Nalpas, A. Mineur, D. Franco, B. Lacour, S. Pol, C. Brechot, P. P. Brechot, Hepatology 2004, 39, 792.
[36] Y. Tang, J. Shi, S. Li, L. Wang, Y. E. Cayre, Y. Chen, Sci Rep. 2014, 2, 1.
[37] W. E. Hennink, C. F. V. Nostrum, Advanced Drug Delivery Reviews 2012, 64, 223.
[38] S. R. Parks, A. M. Bennett, S. E. Speight, J. E. Benbough, J Appl Bacteriol 1996, 80, 529.
[39] S. Engelhart, A. Glasmacher, A. Simon, M. Exner, Int J Hyg Environ Health. 2007, 210, 733.
[40] G. Buckley, Martindale: The Extra Pharmacopeia. J R Coll Gen Pract. 1989, 39, 440.
[41] H. S. Stoker, General, Organic, and Biological Chemistry, 2015.
[42] K. R. Wigginton, P. J. Vikesland, Analyst 2010, 135, 1320.
[43] L. Liang, C. Aiken, R. Mcclelland, L. C. Morrison, N. Tatari, M. Remke, V. Ramaswamy, M. Issaivanan, T. Ryken, M. R. D. Bigio, M. D. Taylor, T. E. Werbowetski-Ogilvie, Oncotarget 2015, 6, 38881.
[44] S. Li, G. C. Poolen, L. C. V. Vliet, J. G. Schipper, R. Broekhuizen, M. Monnikhof, W. V. Hecke, J. F. Vermeulen, N. Bovenschen, Clinical and Translational Oncology 2022, 4, 1204.
[45] R. C. Castellino, B. G. Barwick, M. Schniederjan, M. C. Buss, O. Becher, D. Hambardzumyan, T. J. MacDonald, D. J. Brat, D. L. Durden, PLoS One 2010, 5, e10849.
[46] V. Barak, H. Goike, K. W. Panaretakis, R. Einarsson, Clinical Biochemistry 2004, 37, 529.
[47] S. J. Lee, H. S. Kim, R. Yu, K. R. Lee, T. A. Gardner, C. Jung, M. H. Jeng, F. Yeung, L. Cheng, C. Kao, Molecular Therapy 2002, 6, 415.
[48] A. A. Adams, P. I. Okagbare, J. Feng, M. L. Hupert, D. Patterson, J. Gottert, R. L. McCarley, D. Nikitopoulos, M. C. Murphy, S. A. Soper, J Am Chem Soc. 2008, 130, 8633.
[49] S. B. Cheng, M. Xie, Y. Chen, J. Xiong, Y. Liu, Z. Chen, S. Guo, Y. Shu, M. Wang, B. F. Yuan, W. G. Dong, W. H. Huang, Anal Chem. 2017, 89, 7924.
[50] E. Reategui, N. Aceto, E. J. Lim, J. P. Sullivan, A. E. Jensen, M. Zeinali, J. M. Martel, A. J. Aranyosi, W. Li, S. Castleberry, A. Bardia, L. V. Sequist, D. A. Haber, S. Maheswaran, P. T. Hammond, M. Toner, S. L. Stott, Advanced Materials 2015, 27, 1593.
[51] Yang, Y. et al. Microfluidic device for DNA amplification of single cancer cells isolated from whole blood by self-seeding microwells. Lab on a Chip 2015, 15, 4331-4337.
[52] Nagrath, S. et al. Isolation of rare circulating tumour cells in cancer patients by microchip technology. Nature 2007, 450, 1235-1239.
[53] Stott, S. L. et al. Isolation of circulating tumor cells using a microvortex-generating herringbone-chip. Proc Natl Acad Sci U S A 2010, 107, 18392-18397.
[54] Sarioglu, A. F. et al. A microfluidic device for label-free, physical capture of circulating tumor cell clusters. Nature Methods 2015, 12, 685-691.
[55] Tan, S. J., Yobas, L., Lee, G. Y. H., Ong, C. N. & Lim, C. T. Microdevice for the isolation and enumeration of cancer cells from blood. Biomedical Microdevices 2009, doi: 10.1007/s10544-009-9305-9.
[56] Kovacs, G. T. A., Petersen, K. & Albin, M. Peer Reviewed: Silicon Micromachining: Sensors to Systems. Analytical Chemistry 1996, 68, 407A-412A.
[57] Xia, Y. & Whitesides, G. M. Soft Lithography. Annual Review of Materials Science 1998, 28, 153-184.
[58] Jeon, N. L., Choi, I. S., Xu, B. & Whitesides, G. M. Large-area patterning by vacuum-assisted micromolding. Advanced Materials 1999, 11, 946-950.
[59] van den Bulcke, A. I. et al. Structural and rheological properties of methacrylamide modified gelatin hydrogels. Biomacromolecules 2000, 1, 31-8.
[60] Ramendra, P. et al. Gelatin Biopolymer: A Journey from Micro to Nano. Journal of Pharmacy Research 2014, 8, 1387-1397.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein; however, other combinations of steps, elements, components, and constituents are included, even though not explicitly stated.

### Additional Embodiments

In some aspects, the following embodiments of the present disclosure are also provided:
Embodiment 1. A sacrificial device comprising a hydrogel that forms a sacrificial structure configured to retain and/or separate a targeted agent within an environment,
   wherein the sacrificial device is configured to release the targeted agent when subjected to a releasing condition, the releasing condition being compatible with the targeted agent and deconstructing the sacrificial structure.
Embodiment 2. The sacrificial device of embodiment 1, wherein the sacrificial structure to retain and/or separate a targeted agent comprises a contoured surface that urges the agent to a retaining portion of the sacrificial device when the sacrificial device is in or contacting the environment.
Embodiment 3. The sacrificial device of embodiment 1 or 2, wherein the sacrificial structure to retain and/or separate a targeted agent forms a chamber as a retaining portion, the chamber having one or more inlet regions, wherein a portion of one or more inlet regions is dimensioned or functionalized to allow entry of the targeted agent.
Embodiment 4. The sacrificial device of embodiment 3, wherein the one or more inlet regions have the same size.
Embodiment 5. The sacrificial device of embodiment 3, wherein the one or more inlet regions have 2 or 3 sizes or functionalizations.
Embodiment 6. The sacrificial device of embodiment 1 or 2, wherein the sacrificial structure to retain and/or separate a targeted agent forms a chamber as a retaining portion, the chamber having an inlet region that is dimensioned or functionalized to prevent entry of components of the environment to provide isolation of the targeted agent.
Embodiment 7. The sacrificial device of any one of embodiments 1-6, wherein the hydrogel is biologically compatible.
Embodiment 8. The sacrificial device of any one of embodiments 1-7, wherein the hydrogel is ionically crosslinked.
Embodiment 9. The sacrificial device of embodiment 8, wherein the hydrogel is ionically crosslinked via Mg²⁺ and/or Ca²⁺ ions.
Embodiment 10. The sacrificial device of any one of embodiments 1-9, wherein the hydrogel is heat sensitive, and wherein the releasing condition comprises the sacrificial device being heated.
Embodiment 11. The sacrificial device of embodiment 10, wherein the hydrogel melts upon the sacrificial device being heated.
Embodiment 12. The sacrificial device of embodiment 10 or embodiment 11, wherein heating the sacrificial device occurs at a physiological temperature.
Embodiment 13. The sacrificial device of any one of embodiments 1-9, wherein the hydrogel is light sensitive, and wherein the releasing condition comprises the sacrificial device being contacted with light.
Embodiment 14. The sacrificial device of any one of embodiments 1-9, wherein the releasing condition comprises exposing the sacrificial device to a chemical treatment.
Embodiment 15. The sacrificial device of any one of embodiments 1-15, further comprising a binding agent dispersed within or bound to the hydrogel.
Embodiment 16. The sacrificial device of embodiment 15, wherein the binding agent is covalently linked to the hydrogel.
Embodiment 17. The sacrificial device of embodiment 15, wherein the binding agent is non-covalently linked to the hydrogel.
Embodiment 18. The sacrificial device of any one of embodiments 15-17, wherein the binding agent binds the targeted agent to the hydrogel.
Embodiment 19. The sacrificial device of any one of embodiments 15-18, wherein the binding agent comprises an antibody.
Embodiment 20. The sacrificial device of any one of embodiments 1-19, wherein the targeted agent comprises a biological agent.
Embodiment 21. The sacrificial device of embodiment 20, wherein the biological agent comprises a cell.
Embodiment 22. The sacrificial device of embodiment 20, wherein the biological agent comprises a protein or a nucleic acid.
Embodiment 23. The sacrificial device of any one of embodiments 1-22, wherein the sacrificial device is configured to isolate the targeted agent from a sample.
Embodiment 24. The sacrificial device of embodiment 23, wherein the sample comprises a bodily fluid.
Embodiment 25. The sacrificial device of embodiment 24, wherein the bodily fluid comprises blood plasma, cerebrospinal fluid, or urine.
Embodiment 26. The sacrificial device of any one of embodiments 1-25, wherein the hydrogel comprises gelatin.
Embodiment 27. A filter comprising a sacrificial device of any one of embodiments 1-26.
Embodiment 28. A microfluidic device comprising a sacrificial device of any one of embodiments 1-26.
Embodiment 29. A method of isolating a targeted agent from a sample, the method comprising:
   contacting the sample with a sacrificial device, wherein the sacrificial device binds the targeted agent;
   separating via the sacrificial device the targeted agent from the sample; and
   subjecting the sacrificial device to a releasing condition, whereupon the targeted agent is released and the sacrificial device is completely or substantially sacrificed.
Embodiment 30. The method of embodiment 29, wherein the sacrificial device is at least 50% sacrificed.
Embodiment 31. The method of embodiment 29 or embodiment 30, comprising:
   Filtering the targeted agent from the sample, wherein the sacrificial device is configured as a filter or a separator.
Embodiment 32. The method of any one of embodiments 29-31, wherein separating the sacrificial device from the sample comprises filtering the sample through the sacrificial device.
Embodiment 33. The method of any one of embodiment 29-32, wherein the sacrificial device comprises a hydrogel.
Embodiment 34. The method of embodiment 33, wherein the hydrogel is biologically compatible.
Embodiment 35. The method of embodiment 33 or embodiment 34, wherein the hydrogel comprises gelatin.
Embodiment 36. The method of any one of embodiments 33-35, wherein the hydrogel is ionically crosslinked.
Embodiment 37. The method of embodiment 36, wherein the hydrogel is ionically crosslinked via Mg²⁺ and/or Ca²⁺ ions.
Embodiment 38. The method of any one of embodiments 29-37, wherein subjecting the sacrificial device to a releasing condition comprises heating the vanishing device or an environment of the vanishing device, wherein the releasing condition of the vanishing device is heat sensitive.
Embodiment 39. The method of embodiment 38, wherein the vanishing device completely or substantially melts upon heating the vanishing device.
Embodiment 40. The method of embodiment 38 or embodiment 39, wherein heating the vanishing device occurs at a physiological temperature.
Embodiment 41. The method of any one of embodiments 29-37, wherein subjecting the sacrificial device to a releasing condition comprises illuminating the sacrificial device or an environment of the sacrificial device, wherein the releasing condition of the sacrificial device is light sensitive.
Embodiment 42. The method of any one of embodiments 29-37, wherein subjecting the sacrificial device to a releasing condition comprises contacting the sacrificial device or an environment of the sacrificial device with a reagent, wherein the releasing condition comprises a chemical treatment with the reagent.
Embodiment 43. The method of any one of embodiments 29-42, wherein the sacrificial device binds the targeted agent via a binding agent dispersed within or bound to the sacrificial device.
Embodiment 44. The method of embodiment 43, wherein the binding agent comprises an antibody.
Embodiment 45. The method of any one of embodiment 29-44, wherein the targeted agent comprises a biological agent.
Embodiment 46. The method of embodiment 45, wherein the biological agent comprises a cell.
Embodiment 47. The method of embodiment 45, wherein the biological agent comprises a protein or a nucleic acid.
Embodiment 48. The method of any one of embodiments 29-47, wherein the sample comprises a bodily fluid.
Embodiment 49. The method of embodiment 48, wherein the bodily fluid comprises blood plasma, cerebrospinal fluid, or urine.
Embodiment 50. The method of any one of embodiment 29-49, wherein the sacrificial device includes a feature of any one of embodiment 1-26.
Embodiment 51. A method of fabricating a vanishing device comprising:
   fabricating a first mold, the first mold made in part of silicon and patterned using a silicon micromachining and/or manufacturing process;
   generating a second mold using the first mold;
   generating the vanishing device from the second mold or a third mold derived therefrom.
Embodiment 52. The method of embodiment 51, wherein the second mold comprises a PDMS mold.
Embodiment 53. The method of embodiment 51 or embodiment 52, wherein the first mold includes a contoured surface to form the contour surface of the fabricated vanishing device that urges a biological agent to a retaining portion of the vanishing device when in an environment, the contoured surface of the first mold being dimensioned according to a pre-defined swelling ratio or dimensional adjustments for when the fabricated vanishing device is in the environment.

1. A sacrificial device comprising a hydrogel that forms a sacrificial structure configured to retain and/or separate a targeted agent within an environment,
wherein the sacrificial device is configured to release the targeted agent when subjected to a releasing condition, the releasing condition being compatible with the targeted agent and deconstructing the sacrificial structure.

2. The sacrificial device of claim 1, wherein the sacrificial structure to retain and/or separate a targeted agent comprises a contoured surface that urges the agent to a retaining portion of the sacrificial device when the sacrificial device is in or contacting the environment.

3. The sacrificial device of claim 1, wherein the sacrificial structure to retain and/or separate a targeted agent forms a chamber as a retaining portion, the chamber having one or more inlet regions, wherein a portion of one or more inlet regions is dimensioned or functionalized to allow entry of the targeted agent, or
wherein the sacrificial structure to retain and/or separate a targeted agent forms a chamber as a retaining portion, the chamber having an inlet region that is dimensioned or functionalized to prevent entry of components of the environment to provide isolation of the targeted agent.

4. (canceled)

5. The sacrificial device of claim 1, wherein the hydrogel is biologically compatible.

6. The sacrificial device of claim 1, wherein the hydrogel is ionically crosslinked.

7. The sacrificial device of claim 6, wherein the hydrogel is ionically crosslinked via Mg²⁺ and/or Ca²⁺ ions.

8. The sacrificial device of claim 1, wherein the hydrogel is heat sensitive, and wherein the releasing condition comprises the sacrificial device being heated.

9-10. (canceled)

11. The sacrificial device of claim 1, wherein the hydrogel is light sensitive, and wherein the releasing condition comprises the sacrificial device being contacted with light.

12. The sacrificial device of claim 1, wherein the releasing condition comprises exposing the sacrificial device to a chemical treatment.

13. The sacrificial device of claim 1, further comprising a binding agent dispersed within or bound to the hydrogel.

14. The sacrificial device of claim 13, wherein the binding agent binds the targeted agent to the hydrogel.

15. The sacrificial device of claim 13, wherein the binding agent comprises an antibody.

16. The sacrificial device of claim 1, wherein the targeted agent comprises a biological agent.

17. The sacrificial device of claim 16, wherein the biological agent comprises a cell, a protein, or a nucleic acid.

18. (canceled)

19. The sacrificial device of claim 1, wherein the sacrificial device is configured to isolate the targeted agent from a sample.

20. The sacrificial device of claim 19, wherein the sample comprises a bodily fluid.

21. The sacrificial device of claim 1, wherein the hydrogel comprises gelatin.

22. A filter or microfluidic device comprising a sacrificial device of claim 1.

23. (canceled)

24. A method of isolating a targeted agent from a sample, the method comprising:
contacting the sample with a sacrificial device, wherein the sacrificial device binds the targeted agent;
separating via the sacrificial device the targeted agent from the sample; and
subjecting the sacrificial device to a releasing condition, whereupon the targeted agent is released and the sacrificial device is completely or substantially sacrificed.

25-43. (canceled)

44. A method of fabricating a vanishing device comprising:
fabricating a first mold, the first mold made in part of silicon and patterned using a silicon micromachining and/or manufacturing process;
generating a second mold using the first mold;
generating the vanishing device from the second mold or a third mold derived therefrom.

45. (canceled)
